SEQUENCE LISTING (22 sequences)

SEQ ID NO: 1 - protein, 603 aa, Homo sapiens
MASASTSKYNSHSLENESIKRTSRDGVNRDLTEAVPRLPGETLITDKEVIYICPFNGPIKGRVYITNYRLYLRSLETDSSLILDVPLGVISRIEKMGGATSRGENSYGLDITCKDMRNLRFALKQEGHSRRDMFEILTRYAFPLAHSLPLFAFLNEEKFNVDGWTVYNPVEEYRRQGLPNHHWRITFINKCYELCDTYPALLVVPYRASDDDLRRVATFRSRNRIPVLSWIHPENKTVIVRCSQPLVGMSGKRNKDDEKYLDVIRETNKQISKLTIYDARPSVNAVANKATGGGYESDDAYHNAELFFLDIHNIHVMRESLKKVKDIVYPNVEESHWLSSLESTHWLEHIKLVLTGAIQVADKVSSGKSSVLVHCSDGWDRTAQLTSLAMLMLDSFYRSIEGFEILVQKEWISFGHKFASRIGHGDKNHTDADRSPIFLQFIDCVWQMSKQFPTAFEFNEQFLIIILDHLYSCRFGTFLFNCESARERQKVTERTVSLWSLINSNKEKFKNPFYTKEINRVLYPVASMRHLELWVNYYIRWNPRIKQQQPNPVEQRYMELLALRDEYIKRLEELQLANSAKLSDPPTSPSSPSQMMPHVQTHF

SEQ ID NO: 2 - protein, 116 aa, Mus sp.
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 3 - protein, 15 aa, Artificial Sequence
  Description of Artificial Sequence Synthetic peptide
GGGGSGGGGSGGGGS

SEQ ID NO: 4 - protein, 111 aa, Mus sp.
DIVLTQSPASLAVSLGQRATISCRASKSVSTSSYSYMHWYQQKPGQPPKLLIKYASYLESGVPARFSGSGSGTDFHLNIHPVEEEDAATYYCQHSREFPWTFGGGTKLELK

SEQ ID NO: 5 - DNA, 3452 bp, Homo sapiens
agagggggcggagcagggcccggcagccgagcagcctggcaacggcggtggcgcccggagcccgagagtttccaggatggcttctgcatcaacttctaaatataattcacactccttggagaatgagtctattaagaggacgtctcgagatggagtcaatcgagatctcactgaggctgttcctcgacttccaggagaaacactaatcactgacaaagaagttatttacatatgtcctttcaatggccccattaagggaagagtttacatcacaaattatcgtctttatttaagaagtttggaaacggattcttctctaatacttgatgttcctctgggtgtgatctcgagaattgaaaaaatgggaggcgcgacaagtagaggagaaaattcctatggtctagatattacttgtaaagacatgagaaacctgaggttcgctttgaaacaggaaggccacagcagaagagatatgtttgagatcctcacgagatacgcgtttcccctggctcacagtctgccattatttgcatttttaaatgaagaaaagtttaacgtggatggatggacagtttacaatccagtggaagaatacaggaggcagggcttgcccaatcaccattggagaataacttttattaataagtgctatgagctctgtgacacttaccctgctcttttggtggttccgtatcgtgcctcagatgatgacctccggagagttgcaacttttaggtcccgaaatcgaattccagtgctgtcatggattcatccagaaaataagacggtcattgtgcgttgcagtcagcctcttgtcggtatgagtgggaaacgaaataaagatgatgagaaatatctcgatgttatcagggagactaataaacaaatttctaaactcaccatttatgatgcaagacccagcgtaaatgcagtggccaacaaggcaacaggaggaggatatgaaagtgatgatgcatatcataacgccgaacttttcttcttagacattcataatattcatgttatgcgggaatctttaaaaaaagtgaaggacattgtttatcctaatgtagaagaatctcattggttgtccagtttggagtctactcattggttagaacatatcaagctcgttttgacaggagccattcaagtagcagacaaagtttcttcagggaagagttcagtgcttgtgcattgcagtgacggatgggacaggactgctcagctgacatccttggccatgctgatgttggatagcttctataggagcattgaagggttcgaaatactggtacaaaaagaatggataagttttggacataaatttgcatctcgaataggtcatggtgataaaaaccacaccgatgctgaccgttctcctatttttctccagtttattgattgtgtgtggcaaatgtcaaaacagttccctacagcttttgaattcaatgaacaatttttgattataattttggatcatctgtatagttgccgatttggtactttcttattcaactgtgaatctgctcgagaaagacagaaggttacagaaaggactgtttctttatggtcactgataaacagtaataaagaaaaattcaaaaaccccttctatactaaagaaatcaatcgagttttatatccagttgccagtatgcgtcacttggaactctgggtgaattactacattagatggaaccccaggatcaagcaacaacagccgaatccagtggagcagcgttacatggagctcttagccttacgcgacgaatacataaagcggcttgaggaactgcagctcgccaactctgccaagctttctgatcccccaacttcaccttccagtccttcgcaaatgatgccccatgtgcaaactcacttctgaggggggaccctggcaccgcattagagctcgaaataaaggcgatagctgactttcatttggggcatttgtaaaaagtagattaaaatatttgcctccatgtagaacttgaactaacataatcttaaactcttgaatatgtgccttctagaatacatattacaagaaaactacagggtccacacggcaatcagaagaaaggagctgagatgaggttttggaaaaccctgacacctttaaaaagcagtttttgaaagacaaaatttagatttaatttacgtcttgagaaatactatatatacaatatatattttgtgggcttaattgaaacaacattattttaaaatcaaaggggatatatgtttgtggaatggattttcctgaagctgcttaacagttgctttggattctctaagatgaatccaaatgtgaaagatgcatgttactgccaaaaccaaattgagctcagcttcctaggcattacccaaaagcaaggtgtttaagtaattgccagcttttataccatcatgagtggtgacttaaggagaaatagctgtatagatgagtttttcattatttggaaatttaggggtagaaaatgttttcccctaattttccagagaagcctatttttatatttttaaaaaactgacagggcccagttaaatatgatttgcattttttaaatttgccagttttattttctaaattctttcatgagcttgcctaaaattcggaatggttttcgggttgtggcaaaccccaaagagagcactgtccaaggatgtcgggagcatcctgctgcttaggggaatgttttcgcaaatgttgctctagtcagtccagctcatctgccaaaatgtagggctaccgtcttggatgcatgagctattgctagagcatcatccttagaaatcagtgccccagatgtacatgtgttgagcgtattcttgaaagtattgtgtttatgcatttcaatttcaatggtgttggcttcccctccccaccccacgcgtgcataaaaactggttctacaaatttttacttgaagtaccaggccgtttgctttttcaggttgttttgttttatagtattaagtgaaattttaaatgcacagttctatttgctatctgaactaattcatttattaagtatatttgtaaaagctaaggctcgagttaaaacaatgaagtgttttacaatgatttgtaaaggactatttataactaatatggttttgttttcaatgaattaagaaagattaaatatatctttgtaaattattttatgtcatagtttaattggtctaccaagtaagacatctcaaatacagtagtataatgtatgaattttgtaagtataagaaattttattagacattctcttactttttgtaaatgctgtaaatatttcataaattaacaaagtgtcactccataaaaagaaagctaatactaatagcctaaaagattttgtgaaatttcatgaaaactttttaatggcaataatgactaaagacctgctgtaataaatgtattaactgaaacctaaaaaaaaaaaaaaaaaa

SEQ ID NO: 6 - protein, 603 aa, Mus musculus
MASASASKYNSHSLENESIKKVSQDGVSQDVSETVPRLPGELLITEKEVIYICPFNGPIKGRVYITNYRLYLRSLETDSALILDVPLGVISRIEYMGGATSRGENSYGLDITCKDLRNLRFALKQEGHSRRDMFEILVKHAFPLAHNLPLFAFVNEEKFNVDGWTVYNPVEEYRRQGLPNHHWRISFINKCYELCETYPALLVVPYRTSDDDLRRIATFRSRNRLPVLSWIHPENKMVIMRCSQPLVGMSGKRNKDDEKYLDVIRETNKQTSKLMIYDARPSVNAVANKATGGGYESDDAYQNSELSFLDIHNIHVMRESLKKVKDIVYPNIEESHWLSSLESTHWLEHIKLVLTGAIQVADQVSSGKSSVLVHCSDGWDRTAQLTSLAMLMLDSFYRTIEGFEILVQKEWISFGHKFASRIGHGDKNHADADRSPIFLQFIDCVWQMSKQFPTAFEFNEGFLITVLDHLYSCRFGTFLFNCDSARERQKLTERTVSLWSLINSNKDKFKNPFYTKEINRVLYPVASMRHLELWVNYYIRWNPRVKQQQPNPVEQRYMELLALRDDYIKRLEELQLANSAKLADAPASTSSSSQMVPHVQTHF

SEQ ID NO: 7 - DNA, 3338 bp, Mus musculus
ggtgagttcgctttcttggctgacctggctcggagccgggcattgcggggatccaggattggaaaggttccaggatggcttctgcatcagcatctaagtataattcacactccttggagaatgaatccattaagaaagtgtctcaagatggagtcagtcaggatgtgagtgagactgtccctcggctcccaggggagttactaattactgaaaaagaagttatttacatatgtcctttcaatggccccattaagggaagagtttacatcacaaattatcgtctttatttaagaagtttggaaacggattctgctctaatacttgatgttcctctgggtgtgatatcaagaattgaatatatgggaggcgcgactagtagaggagaaaattcctatggtctagatattacttgtaaagatttgagaaacctgaggtttgcattgaagcaagaaggccacagcagaagagatatgtttgagatccttgtaaaacatgcctttcctctggcacacaatctgccattatttgcatttgtaaatgaagagaagtttaacgtggatgggtggactgtttataatccagttgaagaatatagaaggcagggcctgcccaatcaccattggaggataagttttattaacaagtgctatgagctctgtgagacataccctgctcttttggtggttccctatcggacctcagatgatgatcttaggaggatcgcaacgtttagatcccgaaatcggcttcctgtactgtcgtggattcacccagaaaacaaaatggtcattatgcgctgcagtcagcctcttgtcggtatgagtggtaaaagaaataaagatgacgagaaatacctggatgtgatcagggaaactaacaaacaaacttctaagctcatgatttatgatgcacgacccagtgtaaatgcagtcgccaacaaggcaacaggaggaggatatgaaagtgatgacgcatatcaaaactcagaactttccttcttagacattcataatattcatgttatgcgagaatctttaaaaaaagtgaaagatattgtttatcccaacatagaagaatctcattggttgtccagtttggagtctactcattggttagaacatatcaagcttgttctgaccggtgccattcaagtggcagaccaagtgtcttcaggaaagagctcggtacttgtgcactgcagtgacggatgggacaggaccgctcagctgacatccttggccatgctgatgttggacagcttctacagaactattgaaggctttgagatattggtacagaaagagtggataagttttggccataaatttgcatctagaataggtcatggtgataaaaaccatgctgatgctgatcgatctcctatttttcttcagtttattgactgtgtgtggcagatgtcgaaacagttccccacagcttttgagttcaatgaaggctttttgattaccgttttggatcatctgtatagctgtcgatttggtactttcttattcaactgtgactcggctcgagaaagacagaaacttacagaaagaacagtttctctatggtcgctaattaacagcaataaagacaaattcaaaaaccccttctatacaaaagaaatcaatcgggttttgtatccagttgccagcatgcgtcacttggaactgtgggtgaattattacatccgatggaatcccagggtcaagcagcaacagcccaacccagtggagcagcgttacatggagcttttggccttgcgtgacgattatataaagaggctcgaggaattgcagctggccaactccgccaagcttgctgatgcccccgcttcgacttccagttcgtcacagatggtgccccatgtgcagacgcacttctgaggggactcacttctggcactgcacttgaactctagataagtgaaatagctgactctcattctgggcatgtggacaaagtagatttaaagtgtctgcctccatttagaagttcaactaacatcttagacttttgagtatgtgccttctgtaatacatatcacaagaaatcgatggtgtccgtgtggcaatcataaggaaggagtcaagagggggttctggaaaatcctcatacttttttttacaaagcacttttgcaaagataaaacttaaatttaatttacctctatataaattctacatatacagtatgtattttgtgggcttaattgaaatattattttaaatccaggggggagatttgtttgcaaaatgtattttcctccagctgcttataacagttgctttggattatctaaaattaatccaaatgtgaaagatgggtattactgccaaagccaaattgcactctgcttcttcagcaaattccaagagcaaggcgtttaaataattgccaatttttattttaccataagtggtaaggtaaaaagaaagatgaacatttcatcattttgaatttttgaaaataaaaggttctcccatcatttttcaagagaagcacatttttatattaagaaaaagtgataaggtttgatttttttttccctcaacattctcagctttgctttctaaattatcccatgatttttgtctaacactgagtcatactcaggttgaaggaaacccataaatagcactgtgcgaggagctggctggcttctgctgcttagaggaatatgttcgcaaacatgcctctagtcaattcgccttatctgctgaagtgtaggggcaccgccttgaatggatgagctatggctagagcatctttctttacagtaatgccccaggtgtattctgtttatgtctctctgtttaaatggtgtgcgtgcataaaaacttgctctgcacattattacttgaagtactgggcaatttgctttttcaggttttttttcattttgttttgtagtatgaaatggaattttaaatgcacagttctatttgatatccgaactaattcatttagtaaatatatttgtaaaagctaaagttaaatcaattaatgttttacagtgatttgtaaaggattatttatagctaatatggttttgttttcagtgaattaagagagattacatttatctttgtaaattattttatgtcatagcttaatggcctaccaaatgagacatctcaaatataatagtataatgtatggattttgtaagtataaaaattattagatattcgtttgctttttgtaaacactgtaaatatttcataaattaaaatgtgtcactccataagaagaaaaaactaatactaatagttgacaggaattggtgaaatttcatgaaaatattttcattgcaataaatattaaaagacctgctg

SEQ ID NO: 8 - protein, 553 aa, Rattus norvegicus
MASSSASDCDAHPVERESMRKVSQDGVRQDMSKSGPRLPGESAITDKEVIYICPFSGPVKGRLYITNYRLYLRSLETDLAPILDVPLGVISRIEKMGGVTSRGENSYGLDITCKDLRNLRFALKQEGHSRRDIFDVLTRHAFPLAYNLPLFAFVNEEKFKVDGWAIYNPVEEYRRQGLPDRHWRISFVNQRYELCDTYPALLVVPYRASDDDLRRVATFRSRNRIPVLSWIHPENRAAIMRCSQPLVGVGGKRSRDDERYLDIIRETNKQTSKLTIYDARPGVNAVANKATGGGYEGEDAYPHAELSFLDIHNIHVMRESLRRVRDIVYPHVEEAHWLSSLESTHWLEHIKLLLTGAIRVADKVASGLSSVLVHCSDGWDRTAQLTTLAMLMLDGFYRSIEGFEILVQKEWISFGHKFSSRIGHGDKNHADADRSPIFLQFIDCVWQMTKQFPTAFEFNECFLVAILDHLYSCRFGTFLLNCEAARERQRLAERTVSVWSLINSNKDEFTNPFYARESNRVIYPVTSVRHLELWVNYYIRWNPRIRQQQPHPM

SEQ ID NO: 9 - DNA, 2536 bp, Rattus norvegicus
gcgagcgcgttggcaccagcggcccccggagtctcaggttccaggatggcgtcctcgtcagcctctgactgtgatgcacaccccgtggagcgtgagtccatgaggaaggtgtctcaagatggagtccgtcaggatatgagcaagagtgggcctcgcctcccaggggaatcagccatcactgacaaggaagtcatctacatttgtcccttcagcggccccgtaaagggacgactttacatcaccaattaccgtctctacctgagaagtctggagacggacttggctccgattcttgacgtccccctaggcgtgatatcgagaatagagaaaatgggaggcgtgacgagtcgaggagagaattcctacggccttgatatcacctgcaaagacctgaggaacctgaggttcgctctgaagcaggaaggacacagcaggagggacatctttgacgtcctcaccagacacgccttccccctggcttacaacctgccgttgtttgcattcgtgaacgaggagaagtttaaagtggatggatgggcgatttacaacccggttgaagagtacagaaggcagggcctccccgatcgccattggcggataagtttcgtcaatcagcgctacgagctctgtgacacctaccctgccctcctggtcgtcccctaccgtgcgtccgatgatgacctcagaagagttgcaacctttaggtccagaaaccggattcccgtgctgtcgtggatccacccagagaacagggcggcgatcatgcggtgcagtcagcctctggttggtgtgggcgggaagagaagcagagatgatgagagatacctggacatcatccgggaaaccaataagcagacctcgaagctcacaatttacgatgcgcggcccggcgtcaatgcggtggccaacaaggcaacgggaggcggctatgagggcgaggacgcgtaccctcacgcggagctctccttcctggacatccacaacatccacgtgatgcgggaatccttacggagggtgagggacatcgtgtacccccacgtggaggaagctcattggctgtccagcttggagtccacccattggttagagcacatcaagcttctcctcactggtgccatccgggtcgcagacaaggtggcatcggggctgagttcagtcctcgtgcactgcagtgacggctgggaccggacggctcaactgaccacgctggccatgctgatgctcgatggcttctaccgcagcatcgagggctttgagatcctggtgcagaaggagtggatcagcttcggacacaagttttcatctagaattggccacggtgacaagaaccacgcggatgccgaccgctccccgattttcctgcagttcatcgactgcgtgtggcagatgacgaagcagttccccacagctttcgagttcaacgagtgcttcctggttgccatcttggatcacctgtacagctgccggttcgggactttcttactaaactgtgaggcggcacgggagagacagagactcgcagaaaggacggtgtctgtgtggtccctgatcaacagcaacaaagacgaattcacaaacccgttctacgcaagggagagcaaccgcgtgatctacccggtcaccagcgtgcgccacctggaactgtgggtgaattactacatccggtggaaccccaggatccggcagcagcagccccaccccatgtagcagcgatataatgagctcctggccctgcgtgacgattacatcaagaagctggaggagctgcagctggccacgcccaccaagctcactgactcctccaccccgccttccggttccgcacagatagctccccgcatgcaaactcacttctgagggggttccgggccccaaaccctgaataagtgacgtcaccaacttccgttctgtgcgcttgtgcaaaggggatataaagtctccgcctctgtgtagaagtcgaactaacaccctagaaccttgtgtgacacgtgtgagtgtgcgccttttgtgacgtgtgagtgtgcgatttgtgtgacatgtgtgaatgtgtaccctgtgtgatacgtgcaagtgtgcgccttgtgtaaagttcgtgagtgtgcacctcctgtaacatgttttgcaaggaatctactgcgcttgtgtgccagtcgtgagtacagagtagggggggtcccggaaaaatcctcacacttttttacaaagcgcttgtgcaaagattaaaattaaattatatcaataattatataaattattataattatattgcaaagattaaaaagttaaatttagtttacctctatataaatccagacatacataatatgtactctgtgcgcttaattgaaacgttattttaaatccagaggggagattttttttgtaaaatggatttttcctccagccacttattttgcaaagataaaaaagttaaaataaaagttaaatttaattataaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 10 - protein, 14 aa, Artificial Sequence
  Description of Artificial Sequence Synthetic peptide
GSTSGSGKSSEGKG

SEQ ID NO: 11 - protein, 873 aa, Artificial Sequence
  Description of Artificial Sequence Synthetic polypeptide
DIVLTQSPASLAVSLGQRATISCRASKSVSTSSYSYMHWYQQKPGQPPKLLIKYASYLESGVPARFSGSGSGTDFHLNIHPVEEEDAATYYCQHSREFPWTFGGGTKLELKGGGGSGGGGSGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSEQKLSEEDLGSTSGSGKSSEGKGMASASTSKYNSHSLENESIKRTSRDGVNRDLTEAVPRLPGETLITDKEVIYICPFNGPIKGRVYITNYRLYLRSLETDSSLILDVPLGVISRIEKMGGATSRGENSYGLDITCKDMRNLRFALKQEGHSRRDMFEILTRYAFPLAHSLPLFAFLNEEKFNVDGWTVYNPVEEYRRQGLPNHHWRITFINKCYELCDTYPALLVVPYRASDDDLRRVATFRSRNRIPVLSWIHPENKTVIVRCSQPLVGMSGKRNKDDEKYLDVIRETNKQISKLTIYDARPSVNAVANKATGGGYESDDAYHNAELFFLDIHNIHVMRESLKKVKDIVYPNVEESHWLSSLESTHWLEHIKLVLTGAIQVADKVSSGKSSVLVHCSDGWDRTAQLTSLAMLMLDSFYRSIEGFEILVQKEWISFGHKFASRIGHGDKNHTDADRSPIFLQFIDCVWQMSKQFPTAFEFNEQFLIIILDHLYSCRFGTFLFNCESARERQKVTERTVSLWSLINSNKEKFKNPFYTKEINRVLYPVASMRHLELWVNYYIRWNPRIKQQQPNPVEQRYMELLALRDEYIKRLEELQLANSAKLSDPPTSPSSPSQMMPHVQTHFHHHHHH

SEQ ID NO: 12 - protein, 5 aa, Mus sp.
NYGMH

SEQ ID NO: 13 - protein, 17 aa, Mus sp.
YISSGSSTIYYADTVKG

SEQ ID NO: 14 - protein, 7 aa, Mus sp.
RGLLLDY

SEQ ID NO: 15 - protein, 15 aa, Mus sp.
RASKSVSTSSYSYMH

SEQ ID NO: 16 - protein, 7 aa, Mus sp.
YASYLES

SEQ ID NO: 17 - protein, 9 aa, Mus sp.
QHSREFPWT

SEQ ID NO: 18 - protein, 878 aa, Artificial Sequence
  Description of Artificial Sequence Synthetic polypeptide
SAGIHDIVLTQSPASLAVSLGQRATISCRASKSVSTSSYSYMHWYQQKPGQPPKLLIKYASYLESGVPARFSGSGSGTDFHLNIHPVEEEDAATYYCQHSREFPWTFGGGTKLELKGGGGSGGGGSGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSEQKLSEEDLGSTSGSGKSSEGKGMASASTSKYNSHSLENESIKRTSRDGVNRDLTEAVPRLPGETLITDKEVIYICPFNGPIKGRVYITNYRLYLRSLETDSSLILDVPLGVISRIEKMGGATSRGENSYGLDITCKDMRNLRFALKQEGHSRRDMFEILTRYAFPLAHSLPLFAFLNEEKFNVDGWTVYNPVEEYRRQGLPNHHWRITFINKCYELCDTYPALLVVPYRASDDDLRRVATFRSRNRIPVLSWIHPENKTVIVRCSQPLVGMSGKRNKDDEKYLDVIRETNKQISKLTIYDARPSVNAVANKATGGGYESDDAYHNAELFFLDIHNIHVMRESLKKVKDIVYPNVEESHWLSSLESTHWLEHIKLVLTGAIQVADKVSSGKSSVLVHCSDGWDRTAQLTSLAMLMLDSFYRSIEGFEILVQKEWISFGHKFASRIGHGDKNHTDADRSPIFLQFIDCVWQMSKQFPTAFEFNEQFLIIILDHLYSCRFGTFLFNCESARERQKVTERTVSLWSLINSNKEKFKNPFYTKEINRVLYPVASMRHLELWVNYYIRWNPRIKQQQPNPVEQRYMELLALRDEYIKRLEELQLANSAKLSDPPTSPSSPSQMMPHVQTHFHHHHHH

SEQ ID NO: 19 - protein, 4 aa, Artificial Sequence
  Description of Artificial Sequence Synthetic peptide
AGIH

SEQ ID NO: 20 - protein, 5 aa, Artificial Sequence
  Description of Artificial Sequence Synthetic peptide
SAGIH

SEQ ID NO: 21 - protein, 50 aa, Artificial Sequence
  Description of Artificial Sequence Synthetic polypeptide
GGGGSGGGGSGGGGSGGGGSGGGGSGGGGSGGGGSGGGGSGGGGSGGGGS

SEQ ID NO: 22 - protein, 6 aa, Artificial Sequence
  Description of Artificial Sequence Synthetic 6xHis tag
HHHHHH

(19) United States
(12) Patent Application Publication
(10) Pub. No.: US 2015/0152170 A1
(43) Pub. Date: Jun. 4, 2015

(54) METHODS FOR INCREASING MUSCLE CONTRACTILITY

(71) Applicant: Valerion Therapeutics, LLC, Concord, MA (US)

(72) Inventors: Dustin D. Armstrong, Quincy, MA (US); Michael O'Callaghan, Sherborn, MA (US); Alan H. Beggs, Needham, MA (US); Michael W. Lawlor, Elm Grove, WI (US)

(21) Appl. No.: 14/403,043

(22) PCT Filed: May 23, 2013
(86) PCT No.: PCT/US2013/042471

§ 371 Date: Nov. 21, 2014

Related U.S. Application Data
(60) Provisional application No. 61/729,160, filed on Nov. 21, 2012.
(60) Provisional application No. 61/650,899, filed on May 23, 2012.

(51) Int. Cl. C07K 16/18; C12N 9/16

(52) U.S. Cl. CPC C07K 16/18; C12N 9/16; C07K 2319/00; C07K 2317/24; A61K 2039/505

(57) ABSTRACT

The present disclosure provides methods for increasing muscle contractility in a myotubular myopathy subject following administration of fewer than 20 doses of a chimeric polypeptide that has a myotubularin protein and an internalizing moiety.

### RELATED APPLICATIONS

This application claims the benefit of priority to U.S. provisional application No. 61/650,899, filed May 23, 2012, and application No. 61/729,160, filed Nov. 21, 2012. The disclosures of each of the foregoing applications are hereby incorporated by reference in their entirety.

### BACKGROUND OF THE DISCLOSURE

Myotubular myopathy (MTM) is a rare and severe X-linked muscle disorder that occurs with an estimated incidence of 1 male in every 50,000 births. Myotubular myopathy is a member of a category of diseases referred to as centronuclear myopathies. A cardinal feature of centronuclear myopathies is that the nucleus is positioned in the center of many of the affected individual's muscle cells, rather than in the normal location at the sarcolemma (muscle cell membrane) margins. Although centronuclear myopathies share this characteristic feature, the various diseases have different causes, afflict different patient populations, and have unique disease progression and prognosis.

Myotubular myopathy is caused by a deficiency of the myotubularin 1 (MTM1) protein, a phosphoinositide phosphatase (Buj-Bello A B et al., Human Molecular Genetics, 2008, Vol. 17, No. 14). MTM1 has been shown to play a role in multiple cellular processes, including endosomal trafficking, excitation contraction coupling (ECC), intermediate filament organization, and apoptosis (Tsukita K, 2004, J.Biol. Chem., 279: 13817-24; Buj-Bello et al., 2009, Proc Natl Acad Sci USA, 106: 18763-68; Dowling, et al., 2009, PLoS Genet, 5: 21000372; Al-Qusairi, et al., 2009, Proc Natl Acad Sci USA, 106: 18763-68; Hnia K, 2011, J Clin Invest, 121: 70-85; Razidlo, et al., 2011, J. Biol Chem, 286: 20005-20019). Animal models lacking MTM1 protein display depressed muscle contraction and muscle force development (Al-Qusairi et al.)

At birth MTM patients present with severe hypotonia and respiratory distress and those that survive the neonatal period are often totally or partially dependent upon ventilator support (Taylor G S et al., Proc Natl Acad Sci USA. 2000 Aug. 1; 97(16):8910-5; Bello A B et al., Proc Natl Acad Sci USA. 2002 Nov. 12; 99(23):15060-5; Pierson C R et al., Neuromuscul Disord. 2007 July; 17(7): 562-568; Herman G E et al., THE JOURNAL OF PEDIATRICS VOLUME 134, NUMBER 2). Patients with MTM that do survive the neonatal period exhibit delayed motor milestones and are susceptible to complications such as scoliosis, malocclusion, pyloric stenosis, spherocytosis, and gall and kidney stones, yet linear growth and intelligence are normal and the disease follows a non-progressive course (Herman G E et al., THE JOURNAL OF PEDIATRICS VOLUME 134, NUMBER 2). Muscle biopsy specimens from MTM patients exhibit excessively small fibers with increased numbers of central nuclei and aggregation of organelles within the central regions of many cells, as well as disorganization of the sarcotubular system on electron microscopy (Dowling, et al., 2009, PLoS Genet, 5: 21000372; McEntegart et al., 2002, Neuromuscul Disord, 12: 939-46; Romero et al., 2010, Neuromuscul Disord, 20: 223-28).

The clinical manifestations of myotubular myopathy are muscle weakness, low muscle tone, and the associated disabilities. Pulmonary complications (presumably due to weakness of the muscles responsible for respiration) also occur and, as noted above, many patients who survive are entirely or partially ventilator dependent. Additionally, there is substantial variability in the degree of impairment of patients. In the most severely effected individuals, there is a high incidence of neonatal death. However, other patients survive, and may even maintain independence from ventilator assistance and/or partial or unaided mobility. For example, patients having the recurrent R69C missense mutation may produce a small amount of functional mutant myotubularin, which often leads to a milder and more stable clinical course of MTM through the childhood of the patient.

The average hospital stay for neonatal MTM patients is ˜90 days. However, patients that survive often require long-term ventilatory assistance and in-home care. The cost of basic supportive care, as well as the costs associated with handling the medical complications that often arise in MTM patients, impose a substantial personal and economic burden on patients and families.

### SUMMARY OF THE DISCLOSURE

There is a need for treatments for ameliorating one or more symptoms of myotubular myopathy. For example, there is a need for treatments that increase muscle contractility. Moreover, there is a need for treatments that can be initiated very early (e.g., perinatally or in early infancy), as well as treatments that can be initiated later in life (e.g., in patients older than 5 years old). Such treatment will help address the needs of patients whose symptoms are so severe that today they face very early death, as well as patients whose symptoms have been managed sufficiently for survival, but for whom effective management and amelioration of symptoms has not been achieved. The present disclosure provides methods and compositions for increasing muscle contractility in MTM patients.

The present disclosure provides methods of increasing muscle contractility, force and power (herein termed “contractility”) in a subject having myotubular myopathy by systemically administering to the subject a chimeric polypeptide comprising a myotubularin (MTM1) polypeptide or a bioactive fragment thereof and an internalizing moiety.

In one aspect, the present disclosure provides a method of increasing muscle contractility in a subject having myotubular myopathy, comprising: systemically administering to the subject an amount of a chimeric polypeptide according to a dosing regimen, wherein the chimeric polypeptide comprises: (i) a myotubularin (MTM1) polypeptide, and (ii) an antibody or antibody fragment comprising:

a heavy chain CDR1 having the amino acid sequence of SEQ ID NO: 12,

a heavy chain CDR2 having the amino acid sequence of SEQ ID NO: 13,

a heavy chain CDR3 having the amino acid sequence of SEQ ID NO: 14,

a light chain CDR1 having the amino acid sequence of SEQ ID NO: 15,

a light chain CDR2 having the amino acid sequence of SEQ ID NO: 16, and

a light chain CDR3 having the amino acid sequence of SEQ ID NO: 17,

wherein the administering of less than 20 doses of said chimeric polypeptide is effective to achieve an initial response, wherein the initial response comprises increasing muscle contractility in at least a subset of muscle in said subject by at least 50% relative to that observed prior to initiation of treatment with the chimeric polypeptide. In some embodiments, the initial response comprises increasing muscle contractility in the subject by at least 100%. In other embodiments, the initial response comprises increasing muscle contractility in the subject by at least 200%. In other embodiments, the initial response comprises increasing muscle contractility in the subject by at least 300%. In other embodiments, the initial response comprises increasing muscle contractility in the subject by at least 350%. In other embodiments, the initial response comprises increasing muscle contractility in the subject by at least 450%.

In one aspect, the present disclosure provides a method of increasing muscle contractility in a subject having myotubular myopathy, comprising:

systemically administering to a subject an effective amount of a chimeric polypeptide comprising:

(i) a myotubularin polypeptide and

(ii) an antibody or antibody fragment comprising
a heavy chain CDR1 having the amino acid sequence of SEQ ID NO: 12,
a heavy chain CDR2 having the amino acid sequence of SEQ ID NO: 13,
a heavy chain CDR3 having the amino acid sequence of SEQ ID NO: 14,
a light chain CDR1 having the amino acid sequence of SEQ ID NO: 15,
a light chain CDR2 having the amino acid sequence of SEQ ID NO: 16, and
a light chain CDR3 having the amino acid sequence of SEQ ID NO: 17,
   wherein the subject receives a first dose of said chimeric polypeptide after the subject is 5 years of age. In other embodiments, the subject receives a first dose of said chimeric polypeptide after the subject is 12 years of age. In other embodiments, the subject receives a first dose of said chimeric polypeptide after the subject is 15 years of age. In other embodiments, the subject receives a first dose of said chimeric polypeptide after the subject is 18 years of age.

In one aspect, the present disclosure provides a method of increasing muscle contractility in a subject having myotubular myopathy, comprising:

systemically administering to a subject an effective amount of a chimeric polypeptide comprising:

(i) a myotubularin polypeptide and

(ii) an antibody or antibody fragment comprising
a heavy chain CDR1 having the amino acid sequence of SEQ ID NO: 12,
a heavy chain CDR2 having the amino acid sequence of SEQ ID NO: 13,
a heavy chain CDR3 having the amino acid sequence of SEQ ID NO: 14,
a light chain CDR1 having the amino acid sequence of SEQ ID NO: 15,
a light chain CDR2 having the amino acid sequence of SEQ ID NO: 16, and
a light chain CDR3 having the amino acid sequence of SEQ ID NO: 17,
   wherein the subject receives a first dose of said chimeric polypeptide before the subject is 5 years of age. In some embodiments, the subject receives a first dose of said chimeric polypeptide before the subject is 1 year of age. In some embodiments, the subject receives a first dose of said chimeric polypeptide before the subject is 9 months of age. In some embodiments, the subject receives a first dose of said chimeric polypeptide before the subject is 6 months of age. In some embodiments, the subject receives a first dose of said chimeric polypeptide before the subject is 3 months of age.

In some embodiments, the administration of less than 20 doses of the chimeric polypeptide when utilizing any of the methods described herein is effective to achieve an initial response, wherein the initial response comprises increasing muscle contractility in at least a subset of muscle in said subject by at least 50% relative to that observed prior to initiation of treatment with the chimeric polypeptide. In some embodiments, the initial response comprises increasing muscle contractility in the subject by at least 100%. In other embodiments, the initial response comprises increasing muscle contractility in the subject by at least 200%. In other embodiments, the initial response comprises increasing muscle contractility in the subject by at least 300%. In other embodiments, the initial response comprises increasing muscle contractility in the subject by at least 350%. In other embodiments, the initial response comprises increasing muscle contractility in the subject by at least 450%.

In one aspect, the present disclosure provides a method of increasing muscle contractility in a subject having myotubular myopathy, comprising: systemically administering to the subject 4-20 doses of a chimeric polypeptide comprising:

(i) a myotubularin polypeptide and

(ii) an antibody or antibody fragment comprising
a heavy chain CDR1 having the amino acid sequence of SEQ ID NO: 12,
a heavy chain CDR2 having the amino acid sequence of SEQ ID NO: 13,
a heavy chain CDR3 having the amino acid sequence of SEQ ID NO: 14,
a light chain CDR1 having the amino acid sequence of SEQ ID NO: 15,
a light chain CDR2 having the amino acid sequence of SEQ ID NO: 16, and
a light chain CDR3 having the amino acid sequence of SEQ ID NO: 17,
   wherein prior to said administration of said chimeric polypeptide, said subject has muscle contractility that is less than 5% of muscle contractility in a healthy control subject; and; wherein, following the administration of the 4-20 doses of the chimeric polypeptide, the muscle contractility in the subject is at least 10% of the muscle contractility in the healthy control subject. In other embodiments, the muscle contractility in the subject is at least 15% of the muscle contractility in the healthy control subject following the administration of the 4-20 doses of the chimeric polypeptide. In other embodiments, the muscle contractility in the subject is at least 18% of the muscle contractility in the healthy control subject following the administration of the 4-20 doses of the chimeric polypeptide.

In some embodiments, any of the methods described herein increases skeletal muscle contractility. In some embodiments, the skeletal muscle comprises Type I and/or Type II muscle fibers. In some embodiments, the Type II muscle fibers are Type IIa, Type IIb or Type IIx muscle fibers. In some embodiments, the skeletal muscle is diaphragm, facial, paraspinal, erector spinae, lower limb or upper limb muscle. In some embodiments, the facial muscle is eyelid, jaw, tongue, lips, mouth or throat muscle.

In certain embodiments of any of the foregoing methods, the chimeric polypeptides are formulated as a composition in a pharmaceutically acceptable carrier. In certain embodiments, the chimeric polypeptide is administered parenterally. In some embodiments, the chimeric polypeptide is administered intravenously, intramuscularly or subcutaneously. In certain embodiments, the chimeric polypeptide is administered intravenously via bolus injection or infusion.

Without being bound by theory, the present disclosure is based, in part, on the substantial beneficial impact on muscle contractility attained following administration of just a few doses of chimeric polypeptide. In fact, the benefits were plainly evident by simply observing the treated animals; which were able to move about their cages in previously unobserved ways following just a few treatments with low dose of chimeric polypeptide. Accordingly, in certain embodiments, the disclosure provides methods of increasing muscle contractility by at least a specified level (e.g., at least 50%, 100%, 150%, 200%, etc.) relative to that observed in the subject, prior to administration (an initial response), following administration of an initial number of doses. In other words, an initial response is obtained following administration of 1, 2, 3, 4, 5, 6, 7, 8, 9, 10, 11, 12, 13, 14, 15, 16, 17, 18, 19, or 20 doses. In certain embodiments, the initial response is obtained following administration of less than or equal to 10, 9, 8, 7, 6, or 5 doses. In certain embodiments, the initial response is obtained following administration of less than or equal to 4 doses. In certain embodiments, the initial response of increasing muscle contractility is achieved without a statistically significant increase in muscle size. However, if administration of chimeric polypeptide is continued beyond achieving the initial response, muscle size may also increase.

In some embodiments, the methods disclosed herein comprise administering one or more additional doses of chimeric polypeptide after achieving an initial response. In other words, the disclosure contemplates further administration of chimeric polypeptide following achieving an initial response. For example, if a given patient achieves an initial response following administration of 8 doses of chimeric polypeptide at a particular dosage form, the disclosure contemplates that the patient may be further treated and receive one or more additional doses. These additional doses may exceed 20 doses, and patients may be treated over a period of many years or even their life time. In some embodiments, the administration of one or more additional doses substantially maintains the initial response. In some embodiments, the administration of one or more additional doses provides further improvement relative to the initial response.

In certain embodiments, any of the foregoing methods comprises the administration of at least 6, 10 or 20 doses of the chimeric polypeptide to the subject. In some embodiments, any of the foregoing methods comprises administering one or more additional doses of chimeric polypeptide after achieving an initial response. In some embodiments, any of the foregoing methods comprises administering the chimeric polypeptide to the subject throughout the lifetime of the subject. In some embodiments, any of the foregoing methods comprises administering the chimeric polypeptide to the subject until the subject is asymptomatic for myotubular myopathy. In certain embodiments, any of the foregoing methods comprises administering the chimeric polypeptide to the subject at least once over a two week period, at least once over a one week period, at least twice over a one week period, or at least once a day.

The disclosure provides various methods for increasing muscle contractility in a subject having myotubular myopathy. The instant methods comprise administration of a chimeric polypeptide. The following illustrates numerous exemplary embodiments of chimeric polypeptides for use in the methods of the disclosure. Such embodiments are merely exemplary, and the disclosure contemplates all combinations of these embodiments which each other, as well as with any of the aspects and embodiments disclosed herein. In certain embodiments, the chimeric polypeptide is a fusion protein. In certain embodiments, the chimeric polypeptide has phosphoinositide phosphatase activity. That is, the chimeric polypeptide has the ability to cleave or hydrolyze a phosphorylated phosphoinositide molecule. In certain embodiments, a substrate for the chimeric polypeptide is PI3 or PIP3.

In certain embodiments, any of the foregoing or following MTM1 polypeptides disclosed herein and for use in a chimeric polypeptide further comprise one or more polypeptide portions that enhance one or more of in vivo stability, in vivo half life, uptake/administration, and/or purification. In certain embodiments, any of the foregoing or following MTM1 polypeptides and/or chimeric polypeptides may further include one or more epitope tags. Such epitope tags may be joined to the MTM1 polypeptide and/or the internalizing moiety. When more than one epitope tag is present (e.g., 2, 3, 4) the tags may be the same or different. In other embodiments, the chimeric polypeptide for use in the present methods does not include an epitope tag or includes an epitope tag different from the exemplary tags disclosed herein.

In certain embodiments of any of the foregoing methods, or of any of the aspects and embodiments disclosed herein, the method comprises administering to a subject a chimeric polypeptide that comprises an antibody or antigen binding fragment. In some embodiments, the antibody or antigen binding fragment is a murine, chimeric, humanized, or fully human antibody or antigen binding fragment. In certain embodiments, the antibody or antigen binding fragment is based on the 3E10 antibody. In certain embodiments, the antibody or antigen binding fragment comprises a light chain variable domain and a heavy chain variable domain (VH), and the VH comprises an amino acid sequence at least 95% identical to SEQ ID NO: 2, or a humanized variant thereof. In other embodiments, the antibody or antigen binding fragment comprises a heavy chain variable domain (VH) and a light chain variable domain (VL), and the VL comprises an amino acid sequence at least 95% identical to SEQ ID NO: 4, or a humanized variant thereof. In other embodiments, the antibody or antigen binding fragment comprises a heavy chain variable domain (VH) comprising the amino acid sequence of SEQ ID NO: 2 and a light chain variable domain

(VL) comprising the amino acid sequence of SEQ ID NO: 4, or a humanized variant thereof. As detailed herein, the VH and VL domains may be included as part of a full length antibody or as part of a fragment, such as an scFv. Moreover, the VH and VL domains may be joined by a linker, or may be joined directly. In either case, the VH and VL domains may be joined in either orientation (e.g., with the VL domain N-terminal to the VH domain or with the VH domain N-terminal to the VL domain). In certain embodiments, the VL domain is N-terminal to the VH domain and the two domains are interconnected by a glycine-serine linker. In certain embodiments, including embodiments of any of the foregoing, the antibody or antigen binding fragment (e.g., antibody fragment) comprises

a VH CDR1 having the amino acid sequence of SEQ ID NO: 12;

a VH CDR2 having the amino acid sequence of SEQ ID NO: 13;

a VH CDR3 having the amino acid sequence of SEQ ID NO: 14;

a VL CDR1 having the amino acid sequence of SEQ ID NO: 15;

a VL CDR2 having the amino acid sequence of SEQ ID NO: 16;

a VL CDR3 having the amino acid sequence of SEQ ID NO: 17.

In certain embodiments, the 6 CDRs are present as part of a murine, chimeric, or humanized antibody or antibody fragment, such as an scFv.

In certain embodiments of any of the foregoing or following, the chimeric polypeptide comprises the amino acid sequence set forth in SEQ ID NO: 11, in the presence or absence of one or more epitope tags, or a variant thereof in which the antibody portion is humanized. In certain embodiments, the chimeric polypeptide comprises the amino acid sequence set forth in SEQ ID NO: 18, in the presence or absence of one or more epitope tags, or a variant thereof in which the antibody portion is humanized. In certain embodiments of any of the foregoing or following, the chimeric polypeptide comprises the amino acid sequence of SEQ ID NO: 1 fused, directly or via a linker, to an scFv comprising the 6 CDRs set forth in SEQ ID NOs 12-17. The scFv portion is, in certain embodiments, a murine or humanized antibody fragment comprising the 6 CDRs set forth in SEQ ID NOs 12-17.

In certain embodiments, the chimeric polypeptides for use in the methods disclosed herein may be produced by chemically conjugating the MTM1 polypeptide, or bioactive fragment thereof, to the internalizing moiety. In some embodiments, the chimeric polypeptide may be produced recombinantly to recombinantly conjugate the MTM1 polypeptide, or bioactive fragment thereof, to the internalizing moiety. In certain embodiments, the chimeric polypeptides for use in the claimed method may be conjugated (e.g., chemically or recombinantly) as described herein.

The disclosure contemplates all combinations of any of the foregoing aspects and embodiments, as well as combinations with any of the embodiments set forth in the detailed description and examples.

### BRIEF DESCRIPTION OF THE DRAWINGS

FIG. 1 provides a graph depicting the average results from two in vitro phosphatase experiments in which the phosphatase activity of the 3E10Fv-MTM1 protein was tested in the presence or absence of Ptd(3,5)P₂ substrate. The chimeric MTM1 polypeptide retains phosphoinositide phosphatase activity.

FIG. 2 shows representative tracings of tetanic responses recorded at a frequency of 150 Hz depicting representative maximum force in each treatment group of Mtm1δ4 mice. Note that the vertical axes of all force tracings are on the same scale. Frequency/stress relationships depict the force elicited in each group of animals while accounting for individual muscle cross sectional area when expressing these data.

FIG. 3 shows hematoxylin and eosin (H and E) and NADH staining of injected tibialis anterior muscles from each treatment group of Mtm1δ4 mice. Bar=50 μm for hematoxylin and eosin images and 100 μm for NADH images. H and E staining is provided in the top panels and NADH staining is provided in the bottom panels.

FIG. 4 provides a chart comparing several disease pathology parameters in Mtm1δ4 mice treated under different conditions. Results are based on observations of the hematoxylin and eosin and NADH stained EDL muscles from the different treatment groups of Mtm1δ4 mice.

### DETAILED DESCRIPTION OF THE INVENTION

The disclosure provides methods of increasing muscle contractility by administering to patients chimeric polypeptides of the disclosure (chimeric polypeptides comprising an MTM1 polypeptide and an internalizing moiety). The methods of the disclosure are based on the surprising finding that the administration of only a few doses of an MTM1/internalizing moiety conjugate (3E10Fv-MTM1) to a mouse model of myotubular myopathy was sufficient to significantly increase muscle contractility in treated mice. In fact, this improvement was observed despite the fact that the dosage form used was a relatively low dose (e.g., 20 ul of 0.1 mg/ml; 2 ug total; approximately 0.1 mg/kg). Moreover, the methods of the disclosure are also based on the surprising finding that the increase in muscle contractility in treated mice was observed prior to any significant increase in myofiber size. As such, utilization of the methods of the disclosure is associated with an improvement in muscle contractility and muscle strength that appears to be independent of an increase in myofiber size. In view of these surprising findings, the methods of the disclosure provide an effective means to directly address the depressed muscle contraction that is associated with myotubular myopathy.

### I. MTM1 Polypeptides

As used herein, the MTM1 polypeptides for use in the methods described herein include various splicing isoforms, fusion proteins, and modified forms of the wildtype MTM1 polypeptide. In certain embodiments, a bioactive fragment, variant, or fusion protein of an MTM1 polypeptide comprises an amino acid sequence that is at least 80%, 85%, 90%, 95%, 97%, 98%, 99% or 100% identical to an MTM1 polypeptide (such as the MTM1 polypeptides represented in one or more of SEQ ID NOs: 1, 6, and 8). As used herein, “fragments” are understood to include bioactive fragments or bioactive variants that exhibit “bioactivity” as described herein. That is, bioactive fragments or variants of MTM1 exhibit bioactivity that can be measured and tested. For example, bioactive fragments or variants exhibit the same or substantially the same bioactivity as native (i.e., wild-type, or normal) MTM1 protein, and such bioactivity can be assessed by the ability of the fragment or variant to, e.g., cleave or hydrolyze an endogenous phosphoinositide substrate known in the art, or an artificial phosphoinositide substrate for in vitro assays (i.e., a phosphoinositide phosphatase activity), recruit and/or associate with other proteins such as, for example, the GTPase Rab5, the PI 3-kinase Vps34 or Vps15 (i.e., proper localization), or treat myotubular myopathy. Methods in which to assess any of these criteria are described herein.

The structure and various motifs of the MTM1 polypeptide have been well characterized in the art (see, e.g., Laporte et al., 2003, Human Molecular Genetics, 12(2):R285-R292; Laporte et al., 2002, Journal of Cell Science 15:3105-3117; Lorenzo et al., 2006, 119:2953-2959). As such, in certain embodiments, various bioactive fragments or variants of the MTM1 polypeptides can be designed and identified by screening polypeptides made, for example, recombinantly from the corresponding fragment of the nucleic acid encoding an MTM1 polypeptide. For example, several domains of MTM1 have been shown to be important for its phosphatase activity or localization. To illustrate, these domains include: Glucosyltransferase, Rab-like GTPase Activator and Myotubularins (GRAM; amino acid positions 29-97 or up to 160 of SEQ ID NO: 1), Rac-Induced recruitment Domain (RID; amino acid positions 161-272 of SEQ ID NO: 1), PTP/DSP homology (amino acid positions 273-471 of SEQ ID NO: 1; catalytic cysteine is amino acid 375 of SEQ ID NO: 1), and SET-interacting domain (SID; amino acid positions 435-486 of SEQ ID NO: 1). Accordingly, any combination of such domains may be constructed to identify fragments or variants of MTM1 that exhibit the same or substantially the same bioactivity as native MTM1. Suitable bioactive fragments can be used to make chimeric polypeptides, and such chimeric polypeptides can be used in any of the methods described herein.

Exemplary fragments that may be used as part of a chimeric polypeptide include, for example: about residues 29-486 of SEQ ID NO: 1. Thus, in certain embodiments, the chimeric polypeptides comprises residues 29-486 of SEQ ID NO: 1.

In certain embodiments, the MTM1 portion of the chimeric polypeptide corresponds to the sequence of human MTM 1. For example, the MTM 1 portion of the chimeric polypeptide comprises an amino acid sequence at least 90%, 92%, 95%, 96%, 97%, 98%, 99%, or 100% identical to SEQ ID NO: 1.

In addition, fragments or variants can be chemically synthesized using techniques known in the art such as conventional Merrifield solid phase f-Moc or t-Boc chemistry. The fragments or variants can be produced (recombinantly or by chemical synthesis) and tested to identify those fragments or variants that can function as well as or substantially similarly to a native MTM1 protein, for example, by testing their ability to cleave or hydrolyze a endogenous phosphoinositide substrate or a synthetic phosphoinositide substrate (i.e., phosphoinositide phosphatase activity), recruit and/or associate with other proteins such as, for example, GTPase Rab5, PI 3-kinase hVps34 or hVps15 (i.e., proper localization), or treat myotubular myopathy.

In certain embodiments, the present disclosure contemplates modifying the structure of an MTM1 polypeptide for such purposes as enhancing therapeutic or prophylactic efficacy, or stability (e.g., ex vivo shelf life and resistance to proteolytic degradation in vivo). Such modified MTM1 polypeptides have the same or substantially the same bioactivity as naturally-occurring (i.e., native or wild-type) MTM1 polypeptide. Modified polypeptides can be produced, for instance, by amino acid substitution, deletion, or addition. For instance, it is reasonable to expect, for example, that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains.

This disclosure further contemplates generating sets of combinatorial mutants of an MTM1 polypeptide, as well as truncation mutants, and is especially useful for identifying bioactive variant sequences. Combinatorially-derived variants can be generated which have a selective potency relative to a naturally occurring MTM1 polypeptide. Likewise, mutagenesis can give rise to variants which have intracellular half-lives dramatically different than the corresponding wild-type MTM1 polypeptide. For example, the altered protein can be rendered either more stable or less stable to proteolytic degradation or other cellular process which result in destruction of, or otherwise inactivation of the protein of interest. Such variants can be utilized to alter the MTM1 polypeptide level by modulating their half-life. There are many ways by which the library of potential MTM 1 variants sequences can be generated, for example, from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential polypeptide sequences. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. AG Walton, Amsterdam: Elsevier pp 273-289; Itakura et al., (1984) Annu Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, MTM1 polypeptide variants can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of the MTM1 polypeptide.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of the MTM1 polypeptides. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In certain embodiments, an MTM1 polypeptide may include a peptide and a peptidomimetic. As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). Where no crystal structure of a target molecule is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro, Calif.), contains about 100,000 compounds that are commercially available and also can be searched to identify potential peptidomimetics of the MTM1 polypeptides.

In certain embodiments, an MTM1 polypeptide may further comprise post-translational modifications. Exemplary post-translational protein modifications include phosphorylation, acetylation, methylation, ADP-ribosylation, ubiquitination, glycosylation, carbonylation, sumoylation, biotinylation or addition of a polypeptide side chain or of a hydrophobic group. As a result, the modified MTM1 polypeptides may contain non-amino acid elements, such as lipids, poly- or mono-saccharide, and phosphates. Effects of such non-amino acid elements on the functionality of an MTM1 polypeptide may be tested for its biological activity, for example, its ability to treat myotubular myopathy or ability to cleave phosphoinositides (e.g., PIP3). Given that the native MTM1 polypeptide is glycosylated, in certain embodiments an MTM1 polypeptide used in a chimeric polypeptide according to the present disclosure is glycosylated. In certain embodiments, the level and pattern of glycosylation is the same as or substantially the same as that of the native MTM1 polypeptide. In other embodiments, the level and/or pattern of glycosylation differs from that of the native MTM1 polypeptide (e.g., underglycosylated, overglycosylated, not glycosylated).

In one specific embodiment of the present disclosure, an MTM1 polypeptide may be modified with nonproteinaceous polymers. In one specific embodiment, the polymer is polyethylene glycol (“PEG”), polypropylene glycol, or polyoxyalkylenes, in the manner as set forth in U.S. Pat. Nos. 4,640,835; 4,496,689; 4,301,144; 4,670,417; 4,791,192 or 4,179,337. PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161).

In certain embodiments, fragments or variants of the MTM1 polypeptide will preferably retain at least 50%, 60%, 70%, 80%, 85%, 90%, 95% or 100% of the biological activity associated with the native MTM1 polypeptide. In certain embodiments, fragments or variants of the MTM1 polypeptide have a half-life (t_{1/2}) which is enhanced relative to the half-life of the native protein. For embodiments in which the half-life is enhanced, the half-life of MTM1 fragments or variants is enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of the native MTM1 protein. In some embodiments, the protein half-life is determined in vitro, such as in a buffered saline solution or in serum. In other embodiments, the protein half-life is an in vivo half life, such as the half-life of the protein in the serum or other bodily fluid of an animal. Similarly, any of the foregoing characteristics may be evaluated for MTM1 in the context of a chimeric polypeptide and compared to that of native MTM 1.

In certain aspects, an MTM1 polypeptide may be a fusion protein which further comprises one or more fusion domains. Well known examples of such fusion domains include, but are not limited to, polyhistidine, Glu-Glu, glutathione S transferase (GST), thioredoxin, protein A, protein G, and an immunoglobulin heavy chain constant region (Fc), maltose binding protein (MBP), which are particularly useful for isolation of the fusion proteins by affinity chromatography. For the purpose of affinity purification, relevant matrices for affinity chromatography, such as glutathione-, amylase-, and nickel- or cobalt-conjugated resins are used. Fusion domains also include “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation.

In certain embodiments, the MTM1 polypeptides may contain one or more modifications that are capable of stabilizing the polypeptides. For example, such modifications enhance the in vitro half life of the polypeptides, enhance circulatory half life of the polypeptides or reducing proteolytic degradation of the polypeptides.

It should be noted that any portion of a chimeric polypeptide of the disclosure may be similarly modified, such as with an epitope tag, a PEG moiety or moieties, and the like. In other words, an epitope tag may be to MTM1 and/or the internalizing moiety. Moreover, the chimeric polypeptides may comprise more than one epitope tags, such as 2 epitope tags, or may include 0 epitope tags.

In some embodiments, an MTM1 protein may be a fusion protein with all or a portion of an Fc region of an immunoglobulin. Similarly, in certain embodiments, all or a portion of an Fc region of an immunoglobulin can be used as a linker to link an MTM1 protein to an internalizing moiety. As is known, each immunoglobulin heavy chain constant region comprises four or five domains. The domains are named sequentially as follows: CH1-hinge-CH2-CH3(-CH4). The DNA sequences of the heavy chain domains have cross-homology among the immunoglobulin classes, e.g., the CH2 domain of IgG is homologous to the CH2 domain of IgA and IgD, and to the CH3 domain of IgM and IgE. As used herein, the term, “immunoglobulin Fc region” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In a preferred embodiment the immunoglobulin Fc region comprises at least an immunoglobulin hinge region a CH2 domain and a CH3 domain, and preferably lacks the CH1 domain. In one embodiment, the class of immunoglobulin from which the heavy chain constant region is derived is IgG (Igγ) (γ subclasses 1, 2, 3, or 4). Other classes of immunoglobulin, IgA (Igα), IgD (Igδ), IgE (Igε) and IgM (Igμ), may be used. The choice of appropriate immunoglobulin heavy chain constant regions is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region preferably comprises at least a portion of a hinge domain, and preferably at least a portion of a CH₃ domain of Fc γ or the homologous domains in any of IgA, IgD, IgE, or IgM. Furthermore, it is contemplated that substitution or deletion of amino acids within the immunoglobulin heavy chain constant regions may be useful in the practice of the disclosure. One example would be to introduce amino acid substitutions in the upper CH2 region to create a Fc variant with reduced affinity for Fc receptors (Cole et al. (1997) J. IMMUNOL. 159:3613). One of ordinary skill in the art can prepare such constructs using well known molecular biology techniques.

### II. Internalizing Moieties

The methods disclosed herein contemplate the administration to a subject a chimeric polypeptide that comprises an internalizing moiety. As used herein, the term “internalizing moiety” refers to a moiety capable of interacting with a target tissue or a cell type to effect delivery of the MTM1 polypeptide into the cell (i.e., penetrate desired cell; transport across a cellular membrane; deliver across cellular membranes to, at least, the cytoplasm). In certain embodiments, this disclosure relates to an internalizing moiety which promotes delivery into muscle cells (e.g., skeletal muscle), as well as certain other cell types. This portion promotes entry of the conjugate into cells. Suitable internalizing moieties promote entry via an ENT2 transporter. ENT2 is expressed preferentially in certain cell types, including muscle (skeletal and cardiac). Accordingly, chimeric polypeptides are delivered into cells, but not ubiquitously. Rather, the chimeric polypeptides are delivered with a level of specificity and enrichment for particular tissues, including skeletal muscle.

In some embodiments, an internalizing moiety is the 3E10 antibody, an antibody that binds the same epitope and/or has the same cell penetrating activity and ENT2 mediated mechanism of penetration as 3E10, a variant of 3E10 that binds the same epitope and/or has the same cell penetrating activity and ENT2 mediated mechanism of penetration as 3E10, or an antigen binding fragment of any of the foregoing. In preferred embodiments, the internalizing moiety comprises:

a VH CDR1 having the amino acid sequence of SEQ ID NO: 12;

a VH CDR2 having the amino acid sequence of SEQ ID NO: 13;

a VH CDR3 having the amino acid sequence of SEQ ID NO: 14;

a VL CDR1 having the amino acid sequence of SEQ ID NO: 15;

a VL CDR2 having the amino acid sequence of SEQ ID NO: 16; and

a VL CDR3 having the amino acid sequence of SEQ ID NO: 17.

In certain aspects, an internalizing moiety may comprise an antibody, including a monoclonal antibody, a polyclonal antibody, and a humanized antibody. Without being bound by theory, such antibody may bind to an antigen of a target tissue and thus mediate delivery to the target tissue (e.g., muscle, cancer cells, etc.). In some embodiments, internalizing moieties may comprise antibody fragments, derivatives or analogs thereof, including without limitation: Fv fragments, single chain Fv (scFv) fragments, Fab′ fragments, F(ab′)2 fragments, single domain antibodies, camelized antibodies and antibody fragments, humanized antibodies and antibody fragments, human antibodies and antibody fragments, and multivalent versions of the foregoing; multivalent internalizing moieties including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments; receptor molecules which naturally interact with a desired target molecule. In certain embodiments, the antibodies or variants thereof, may be modified to make them less immunogenic when administered to a subject. For example, if the subject is human, the antibody may be “humanized”, for example as described in Jones, P. et al. (1986), Nature, 321, 522-525 or Tempest et al. (1991), Biotechnology, 9, 266-273. In some embodiments, the internalizing moiety is any peptide or antibody-like protein having the complementarity determining regions (CDRs) of the 3E10 antibody sequence, or of an antibody that binds the same epitope as 3E10, such as the six CDRs set forth in SEQ ID NOs 12-17.

In certain embodiments, the internalizing moiety comprises the monoclonal antibody 3E10 or an antigen binding fragment thereof. For example, the antibody or antigen binding fragment thereof may be monoclonal antibody 3E10, or a variant thereof that retains the cell penetrating activity of 3E10, or an antigen binding fragment of 3E10 or said 3E10 variant. Additionally, the antibody or antigen binding fragment thereof may be an antibody that binds to the same epitope as 3E10, or an antibody that has substantially the same cell penetrating activity as 3E10, or an antigen binding fragment thereof. These are exemplary of agents that target ENT2. In certain embodiments, the antigen binding fragment (also referred to as the antibody fragment) is an Fv or scFv fragment thereof. Monoclonal antibody 3E10 can be produced by a hybridoma 3E10 placed permanently on deposit with the American Type Culture Collection (ATCC) under ATCC accession number PTA-2439 and is disclosed in U.S. Pat. No. 7,189,396. Additionally or alternatively, the 3E10 antibody can be produced by expressing in a host cell nucleotide sequences encoding the heavy and light chains of the 3E10 antibody. The term “3E10 antibody” or “monoclonal antibody 3E10” are used to refer to the antibody, regardless of the method used to produce the antibody. Similarly, when referring to variants or antigen-binding fragments of 3E10, such terms are used without reference to the manner in which the antibody was produced. At this point, 3E10 is generally not produced by the hybridoma but is produced recombinantly. Thus, in the context of the present application, 3E10 antibody will refer to an antibody comprising a variable heavy chain domain comprising the amino acid sequence set forth in SEQ ID NO: 2 and the variable light chain domain comprising the amino acid sequence set forth in SEQ ID NO: 4.

The internalizing moiety may also comprise variants of mAb 3E10, such as variants of 3E10 which retain the same cell penetration characteristics as mAb 3E10, as well as variants modified by mutation to improve the utility thereof (e.g., improved ability to target specific cell types, improved ability to penetrate the cell membrane, improved ability to localize to the cellular DNA, convenient site for conjugation, and the like). Such variants include variants wherein one or more conservative substitutions are introduced into the heavy chain, the light chain and/or the constant region(s) of the antibody. Such variants include humanized versions of 3E10 or a 3E10 variant. In some embodiments, the light chain or heavy chain may be modified at the N-terminus or C-terminus. Moreover, the antibody or antibody fragment may be modified to facilitate conjugation to an MTM1 polypeptide. Similarly, the foregoing description of variants applies to antigen binding fragments. Any of these antibodies, variants, or fragments may be made recombinantly by expression of the nucleotide sequence(s) in a host cell.

Monoclonal antibody 3E10 has been shown to penetrate cells to deliver proteins and nucleic acids into the cytoplasmic or nuclear spaces of target tissues (Weisbart R H et al., J Autoimmun. 1998 October; 11(5):539-46; Weisbart R H, et al. Mol Immunol. 2003 March; 39(13):783-9; Zack D J et al., J Immunol. 1996 Sep. 1; 157(5):2082-8.). Further, the VH and Vk sequences of 3E10 are highly homologous to human antibodies, with respective humanness z-scores of 0.943 and −0.880. Thus, Fv3E10 is expected to induce less of an anti-antibody response than many other approved humanized antibodies (Abhinandan K R et al., Mol. Biol. 2007 369, 852-862). A single chain Fv fragment of 3E10 possesses all the cell penetrating capabilities of the original monoclonal antibody, and proteins such as catalase, dystrophin, HSP70 and p53 retain their activity following conjugation to Fv3E10 (Hansen J E et al., Brain Res. 2006 May 9; 1088(1):187-96; Weisbart R H et al., Cancer Lett. 2003 Jun. 10; 195(2):211-9; Weisbart R H et al., J Drug Target. 2005 February; 13(2):81-7; Weisbart R H et al., J Immunol. 2000 Jun. 1; 164(11):6020-6; Hansen J E et al., J Biol Chem. 2007 Jul. 20; 282(29):20790-3). The 3E10 is built on the antibody scaffold present in all mammals; a mouse variable heavy chain and variable kappa light chain. 3E10 gains entry to cells via the ENT2 nucleotide transporter that is particularly enriched in skeletal muscle and cancer cells, and in vitro studies have shown that 3E10 is nontoxic. (Weisbart R H et al., Mol Immunol. 2003 March; 39(13):783-9; Pennycooke M et al., Biochem Biophys Res Commun. 2001 Jan. 26; 280(3):951-9).

The internalizing moiety may also include mutants of mAb 3E10, such as variants of 3E10 which retain the same or substantially the same cell penetration characteristics as mAb 3E10, as well as variants modified by mutation to improve the utility thereof (e.g., improved ability to target specific cell types, improved ability to penetrate the cell membrane, improved ability to localize to the cellular DNA, improved binding affinity, and the like). Such mutants include variants wherein one or more conservative substitutions are introduced into the heavy chain, the light chain. Numerous variants of mAb 3E10 have been characterized in, e.g., U.S. Pat. No. 7,189,396 and WO 2008/091911, the teachings of which are incorporated by reference herein in their entirety.

In certain embodiments, the internalizing moiety comprises an antibody or antigen binding fragment comprising an VH domain comprising an amino acid sequence at least 80%, 85%, 90%, 95%, 96%, 97%, 99%, or 100% identical to SEQ ID NO: 2 and/or a VL domain comprising an amino acid sequence at least 85%, 90%, 95%, 96%, 97%, 99%, or 100% identical to SEQ ID NO: 4. Of course, such internalizing moieties transit cells via ENT2 and/or bind the same epitope as 3E10. In certain embodiments, such an internalizing moiety is a humanized variant of any of the foregoing (e.g., in other words, an antibody or antibody fragment having these heavy or light chains may then further be humanized).

In certain embodiments, the internalizing moiety is an antigen binding fragment, such as a single chain Fv of 3E10 (scFv) comprising SEQ ID NOs: 2 and 4). In certain embodiments, the internalizing moiety comprises a single chain Fv of 3E10 (or another antigen binding fragment), and the amino acid sequence of the V_{H} domain is at least 90%, 95%, 96%, 97%, 98%, 99%, or 100% identical to SEQ ID NO: 2, and amino acid sequence of the V_{L} domain is at least 90%, 95%, 96%, 97%, 98%, 99%, or 100% identical to SEQ ID NO: 4. The variant 3E10 or fragment thereof retains the function of an internalizing moiety and transit cells via ENT2 and/or bind the same epitope as 3E10. In certain embodiments, such an internalizing moiety is a humanized variant of any of the foregoing (e.g., in other words, an antibody or antibody fragment having these heavy or light chains may then further be humanized).

In certain embodiments, the internalizing moiety comprises at least 1, 2, 3, 4, or 5 of the CDRs of 3E10 (e.g., which are set forth in SEQ ID NOs: 12-17). In certain embodiments, the internalizing moiety comprises all six CDRs of 3E10 (e.g., comprises SEQ ID NOs 12-17). Such an antibody or antibody fragment may be a humanized antibody or antibody fragment. For any of the foregoing, in certain embodiments, the internalizing moiety is an antibody that binds the same epitope as 3E10 and/or the internalizing moiety competes with 3E10 for binding to antigen. Exemplary internalizing moieties target and transit cells via ENT2.

The present disclosure utilizes the cell penetrating ability of 3E10 or 3E10 fragments or variants to promote delivery of MTM1 in vivo. 3E10 and 3E10 variants and fragments are particularly well suited for this because of their demonstrated ability to effectively promote delivery to muscle cells, including skeletal, as well as diaphragm. Thus, 3E10 and 3E10 variants and fragments are especially useful for promoting effective delivery into cells in subjects, such as human patients or model organisms, having MTM or symptoms that recapitulate MTM.

As described further below, a recombinant 3E10 or 3E10-like variant or fragment can be conjugated, linked or otherwise joined to an MTM1 polypeptide. Methods of chemically conjugating polypeptides to other polypeptides are well known in the art and include, addition of a free cysteine to the C-terminus of, for example, an scFv or other antigen-binding fragment to generate a site for site-directed conjugation. In the context of making chimeric polypeptides to MTM1, chemical conjugation, as well as making the chimeric polypeptide as a fusion protein is available and known in the art.

Preparation of antibodies or fragments thereof (e.g., a single chain Fv fragment encoded by V_{H}-linker-V_{L} or V_{L}-linker-V_{H}) is well known in the art. In particular, methods of recombinant production of mAb 3E10 antibody fragments have been described in WO 2008/091911. Further, methods of generating scFv fragments of antibodies are well known in the art. When recombinantly producing an antibody or antibody fragment, a linker may be used. For example, typical surface amino acids in flexible protein regions include Gly, Asn and Ser. One exemplary linker is provided in SEQ ID NO: 15. Permutations of amino acid sequences containing Gly, Asn and Ser would be expected to satisfy the criteria (e.g., flexible with minimal hydrophobic or charged character) for a linker sequence. Another exemplary linker is of the formula (G₄S)n, wherein n is an integer from 1-10, such as 2, 3, or 4. Other near neutral amino acids, such as Thr and Ala, can also be used in the linker sequence.

In addition to linkers interconnecting portions of, for example, an scFv, the disclosure contemplates the use of additional linkers to, for example, interconnect the MTM1 polypeptide to the antibody portion of the chimeric polypeptide or to interconnect the MTM1 portion to the antibody portion of the chimeric polypeptide.

Preparation of antibodies may be accomplished by any number of well-known methods for generating monoclonal antibodies. These methods typically include the step of immunization of animals, typically mice, with a desired immunogen (e.g., a desired target molecule or fragment thereof). Once the mice have been immunized, and preferably boosted one or more times with the desired immunogen(s), monoclonal antibody-producing hybridomas may be prepared and screened according to well known methods (see, for example, Kuby, Janis, Immunology, Third Edition, pp. 131-139, W.H. Freeman & Co. (1997), for a general overview of monoclonal antibody production, that portion of which is incorporated herein by reference). Over the past several decades, antibody production has become extremely robust. In vitro methods that combine antibody recognition and phage display techniques allow one to amplify and select antibodies with very specific binding capabilities. See, for example, Holt, L. J. et al., “The Use of Recombinant Antibodies in Proteomics,” Current Opinion in Biotechnology, 2000, 11:445-449, incorporated herein by reference. These methods typically are much less cumbersome than preparation of hybridomas by traditional monoclonal antibody preparation methods. In one embodiment, phage display technology may be used to generate an internalizing moiety specific for a desired target molecule. An immune response to a selected immunogen is elicited in an animal (such as a mouse, rabbit, goat or other animal) and the response is boosted to expand the immunogen-specific B-cell population. Messenger RNA is isolated from those B-cells, or optionally a monoclonal or polyclonal hybridoma population. The mRNA is reverse-transcribed by known methods using either a poly-A primer or murine immunoglobulin-specific primer(s), typically specific to sequences adjacent to the desired V_{H} and V_{L} chains, to yield cDNA. The desired V_{H} and V_{L} chains are amplified by polymerase chain reaction (PCR) typically using V_{H} and V_{L} specific primer sets, and are ligated together, separated by a linker. V_{H} and V_{L} specific primer sets are commercially available, for instance from Stratagene, Inc. of La Jolla, Calif. Assembled V_{H}-linker-V_{L} product (encoding an scFv fragment) is selected for and amplified by PCR. Restriction sites are introduced into the ends of the V_{H}-linker-V_{L} product by PCR with primers including restriction sites and the scFv fragment is inserted into a suitable expression vector (typically a plasmid) for phage display. Other fragments, such as an Fab′ fragment, may be cloned into phage display vectors for surface expression on phage particles. The phage may be any phage, such as lambda, but typically is a filamentous phage, such as fd and M13, typically M13.

In certain embodiments, an antibody or antibody fragment is made recombinantly in a host cell. In other words, once the sequence of the antibody is known (for example, using the methods described above), the antibody can be made recombinantly using standard techniques.

In certain embodiments, the antibody or antibody fragment is humanized. Methods of humanizing an antibody or antibody fragment are well known in the art. Given, for example, the amino acid sequence of a VH and/or VL, one of skill in the art can generate a humanized antibody or antibody fragment. Thus, the disclosure includes the use of chimeric polypeptides in which the antibody fragment portion of the chimeric polypeptide is humanized.

In certain embodiments, the internalizing moieties may be modified to make them more resistant to cleavage by proteases. For example, the stability of an internalizing moiety comprising a polypeptide may be increased by substituting one or more of the naturally occurring amino acids in the (L) configuration with D-amino acids. In various embodiments, at least 1%, 5%, 10%, 20%, 50%, 80%, 90% or 100% of the amino acid residues of internalizing moiety may be of the D configuration. The switch from L to D amino acids neutralizes the digestion capabilities of many of the ubiquitous peptidases found in the digestive tract. Alternatively, enhanced stability of an internalizing moiety comprising an peptide bond may be achieved by the introduction of modifications of the traditional peptide linkages. For example, the introduction of a cyclic ring within the polypeptide backbone may confer enhanced stability in order to circumvent the effect of many proteolytic enzymes known to digest polypeptides in the stomach or other digestive organs and in serum. In still other embodiments, enhanced stability of an internalizing moiety may be achieved by intercalating one or more dextrorotatory amino acids (such as, dextrorotatory phenylalanine or dextrorotatory tryptophan) between the amino acids of internalizing moiety. In exemplary embodiments, such modifications increase the protease resistance of an internalizing moiety without affecting the activity or specificity of the interaction with a desired target molecule.

### III. Chimeric Polypeptides

Chimeric polypeptides for use in the present disclosure can be made in various manners. In certain embodiments, the C-terminus of an MTM1 polypeptide can be linked to the N-terminus of an internalizing moiety. Alternatively, the C-terminus of an internalizing moiety can be linked to the N-terminus of an MTM1 polypeptide. For example, chimeric polypeptides can be designed to place the MTM1 polypeptide at the amino or carboxy terminus of either the antibody heavy or light chain of 3E10. In certain embodiments, potential configurations include the use of truncated portions of an antibody's heavy and light chain sequences (e.g., 3E10) as needed to maintain the functional integrity of the attached MTM1 polypeptide. Further still, the internalizing moiety can be linked to an exposed internal (non-terminus) residue of MTM1 or a variant thereof. In further embodiments, any combination of the MTM1-internalizing moiety configurations can be employed, thereby resulting in an MTM1:internalizing moiety ratio that is greater than 1:1 (e.g., two MTM1 molecules to one internalizing moiety).

In certain embodiments, the chimeric polypeptides for use in the present disclosure comprise the amino acid sequence set forth in SEQ ID NO: 11, in the presence or absence of one or more epitope tags, or a variant thereof in which the antibody portion is humanized (e.g., the antibody fragment portion of this chimeric polypeptide is humanized). In certain embodiments, the chimeric polypeptides comprise a “AGIH” portion (SEQ ID NO: 19) on the N-terminus of the polypeptide, and such chimeric polypeptides may be provided in the presence or absence of one or more epitope tags. In further embodiments, the chimeric polypeptide comprises a serine at the N-terminal most position of the polypeptide. In some embodiments, the chimeric polypeptides comprise an “SAGIH” (SEQ ID NO: 20) portion at the N-terminus of the polypeptide, and such chimeric polypeptides may be provided in the presence or absence of one or more epitope tags. In certain embodiments, the chimeric polypeptide comprises the amino acid sequence set forth in SEQ ID NO: 18, in the presence or absence of one or more epitope tags, or a variant thereof in which the antibody portion is humanized (e.g., the antibody fragment portion of this chimeric polypeptide is humanized).

The MTM1 polypeptide and the internalizing moiety may be conjugated directly to each other. Alternatively, they may be linked to each other via a linker sequence, which separates the MTM1 polypeptide and the internalizing moiety by a distance sufficient to ensure that each domain properly folds into its secondary and tertiary structures. Preferred linker sequences (1) should adopt a flexible extended conformation, (2) should not exhibit a propensity for developing an ordered secondary structure which could interact with the functional domains of the MTM1 polypeptide or the internalizing moiety, and (3) should have minimal hydrophobic or charged character, which could promote interaction with the functional protein domains. Typical surface amino acids in flexible protein regions include Gly, Asn and Ser. Permutations of amino acid sequences containing Gly, Asn and Ser would be expected to satisfy the above criteria for a linker sequence. Other near neutral amino acids, such as Thr and Ala, can also be used in the linker sequence. In a specific embodiment, a linker sequence length of about 15 amino acids can be used to provide a suitable separation of functional protein domains, although longer or shorter linker sequences may also be used. The length of the linker sequence separating the MTM1 polypeptide and the internalizing moiety can be from 5 to 500 amino acids in length, or more preferably from 5 to 100 amino acids in length. Preferably, the linker sequence is from about 5-30 amino acids in length. In preferred embodiments, the linker sequence is from about 5 to about 20 amino acids, and is advantageously from about 10 to about 20 amino acids. In other embodiments, the linker joining the MTM1 polypeptide to an internalizing moiety can be a constant domain of an antibody (e.g., constant domain of Ab 3E10 or all or a portion of an Fc region of another antibody). By way of example, the linker that joins MTM1 with an internalizing moiety is GSTSGSGKSSEGKG (SEQ ID NO: 10). In certain embodiments, the linker is a cleavable linker. As noted above, the chimeric polypeptide may include more than one linker, such as a linker joining the internalizing moiety to the MTM polypeptide and a linker joining portions of the internalizing moiety to each other (e.g., a linker joining a VH and VL domain of a single chain Fv fragment). When the chimeric polypeptide includes more than one linker, such as two linkers, the linkers are independently selected and may be the same or different.

In certain embodiments, the chimeric polypeptides for use in the methods of the present disclosure can be generated using well-known cross-linking reagents and protocols. For example, there are a large number of chemical cross-linking agents that are known to those skilled in the art and useful for cross-linking the MTM1 polypeptide with an internalizing moiety (e.g., an antibody). For example, the cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including succinimidyl 4-(N-maleimidomethyl) cyclohexane-1-carboxylate (SMCC), m-Maleimidobenzoyl-N-hydroxysuccinimide ester (MBS); N-succinimidyl (4-iodoacetyl) aminobenzoate (SIAB), succinimidyl 4-(p-maleimidophenyl) butyrate (SMPB), 1-ethyl-3-(3-dimethylaminopropyl) carbodiimide hydrochloride (EDC); 4-succinimidyloxycarbonyl-a-methyl-a-(2-pyridyldithio)-tolune (SMPT), N-succinimidyl 3-(2-pyridyldithio) propionate (SPDP), succinimidyl 6-[3-(2-pyridyldithio) propionate] hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo. In addition to the heterobifunctional cross-linkers, there exists a number of other cross-linking agents including homobifunctional and photoreactive cross-linkers. Disuccinimidyl subcrate (DSS), bismaleimidohexane (BMH) and dimethylpimelimidate. 2 HCl (DMP) are examples of useful homobifunctional cross-linking agents, and bis-[B-(4-azidosalicylamido)ethyl]disulfide (BASED) and N-succinimidyl-6(4′-azido-2′-nitrophenylamino)hexanoate (SANPAH) are examples of useful photoreactive cross-linkers for use in this disclosure. For a recent review of protein coupling techniques, see Means et al. (1990) Bioconjugate Chemistry. 1:2-12, incorporated by reference herein.

One particularly useful class of heterobifunctional cross-linkers, included above, contain the primary amine reactive group, N-hydroxysuccinimide (NHS), or its water soluble analog N-hydroxysulfosuccinimide (sulfo-NHS). Primary amines (lysine epsilon groups) at alkaline pH's are unprotonated and react by nucleophilic attack on NHS or sulfo-NHS esters. This reaction results in the formation of an amide bond, and release of NHS or sulfo-NHS as a by-product. Another reactive group useful as part of a heterobifunctional cross-linker is a thiol reactive group. Common thiol reactive groups include maleimides, halogens, and pyridyl disulfides. Maleimides react specifically with free sulfhydryls (cysteine residues) in minutes, under slightly acidic to neutral (pH 6.5-7.5) conditions. Halogens (iodoacetyl functions) react with —SH groups at physiological pH's. Both of these reactive groups result in the formation of stable thioether bonds. The third component of the heterobifunctional cross-linker is the spacer arm or bridge. The bridge is the structure that connects the two reactive ends. The most apparent attribute of the bridge is its effect on steric hindrance. In some instances, a longer bridge can more easily span the distance necessary to link two complex biomolecules.

Preparing protein-conjugates using heterobifunctional reagents is a two-step process involving the amine reaction and the sulfhydryl reaction. For the first step, the amine reaction, the protein chosen should contain a primary amine. This can be lysine epsilon amines or a primary alpha amine found at the N-terminus of most proteins. The protein should not contain free sulfhydryl groups. In cases where both proteins to be conjugated contain free sulfhydryl groups, one protein can be modified so that all sulfhydryls are blocked using for instance, N-ethylmaleimide (see Partis et al. (1983) J. Pro. Chem. 2:263, incorporated by reference herein). Ellman's Reagent can be used to calculate the quantity of sulfhydryls in a particular protein (see for example Ellman et al. (1958) Arch. Biochem. Biophys. 74:443 and Riddles et al. (1979) Anal. Biochem. 94:75, incorporated by reference herein).

In certain specific embodiments, chimeric polypeptides for use in the methods of the present disclosure can be produced by using a universal carrier system. For example, an MTM1 polypeptide can be conjugated to a common carrier such as protein A, poly-L-lysine, hex-histidine, and the like. The conjugated carrier will then form a complex with an antibody which acts as an internalizing moiety. A small portion of the carrier molecule that is responsible for binding immunoglobulin could be used as the carrier.

In certain embodiments, chimeric polypeptides for use in the methods of the present disclosure can be produced by using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). In any of the foregoing methods of cross-linking for chemical conjugation of MTM1 to an internalizing moiety, a cleavable domain or cleavable linker can be used. Cleavage will allow separation of the internalizing moiety and the MTM1 polypeptide. For example, following penetration of a cell by a chimeric polypeptide, cleavage of the cleavable linker would allow separation of MTM 1 from the internalizing moiety.

In certain embodiments, the chimeric polypeptides for use in the methods of the present disclosure are generated as a fusion protein containing a MTM1 polypeptide and an internalizing moiety, expressed as one contiguous polypeptide chain. Such chimeric polypeptides are referred to herein as recombinantly conjugated. In preparing such fusion proteins, a fusion gene is constructed comprising nucleic acids which encode an MTM1 polypeptide and an internalizing moiety, and optionally, a peptide linker sequence to span the MTM1 polypeptide and the internalizing moiety. Alternatively, one or more portions of the chimeric polypeptide may be recombinantly produced separately, and the portions may be later combined chemically or recombinantly. The use of recombinant DNA techniques to create a fusion gene, with the translational product being the desired fusion protein, is well known in the art. Both the coding sequence of a gene and its regulatory regions can be redesigned to change the functional properties of the protein product, the amount of protein made, or the cell type in which the protein is produced. The coding sequence of a gene can be extensively altered--for example, by fusing part of it to the coding sequence of a different gene to produce a novel hybrid gene that encodes a fusion protein. Examples of methods for producing fusion proteins are described in PCT applications PCT/US87/02968, PCT/US89/03587 and PCT/US90/07335, as well as Traunecker et al. (1989) Nature 339:68, incorporated by reference herein. Essentially, the joining of various DNA fragments coding for different polypeptide sequences is performed in accordance with conventional techniques, employing blunt-ended or stagger-ended termini for ligation, restriction enzyme digestion to provide for appropriate termini, filling in of cohesive ends as appropriate, alkaline phosphatase treatment to avoid undesirable joining, and enzymatic ligation. Alternatively, the fusion gene can be synthesized by conventional techniques including automated DNA synthesizers. In another method, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, Eds. Ausubel et al. John Wiley & Sons: 1992). The chimeric polypeptides encoded by the fusion gene may be recombinantly produced using various expression systems as is well known in the art (also see below).

Recombinantly conjugated chimeric polypeptides include embodiments in which the MTM1 polypeptide is conjugated to the N-terminus or C-terminus of the internalizing moiety.

In some embodiments, the immunogenicity of the chimeric polypeptide may be reduced by identifying a candidate T-cell epitope within a junction region spanning the chimeric polypeptide and changing an amino acid within the junction region as described in U.S. Patent Publication No. 2003/0166877.

### IV. MTM1-Related Nucleic Acids and Expression

In certain embodiments, the present disclosure makes use of nucleic acids for producing an MTM1 polypeptide or a chimeric polypeptide for use in any of the methods described herein. In certain specific embodiments, the nucleic acids may further comprise DNA which encodes an internalizing moiety for making a recombinant chimeric protein of the invention. All these nucleic acids are collectively referred to as MTM1 nucleic acids.

The nucleic acids may be single-stranded or double-stranded, DNA or RNA molecules. In certain embodiments, the disclosure relates to isolated or recombinant nucleic acid sequences that are at least 80%, 85%, 90%, 95%, 97%, 98%, 99% or 100% identical to a region of an MTM1 nucleotide sequence (e.g., SEQ ID NOs: 5, 7, and 9). In further embodiments, the MTM1 nucleic acid sequences can be isolated, recombinant, and/or fused with a heterologous nucleotide sequence, or in a DNA library.

In certain embodiments, MTM1 nucleic acids also include nucleotide sequences that hybridize under highly stringent conditions to any of the above-mentioned native MTM1 nucleotide sequence, or complement sequences thereof. One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the native MTM1 nucleic acids due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant MTM1 and/or chimeric polypeptide encoding nucleic acids may be operably linked to one or more regulatory nucleotide sequences in an expression construct. Regulatory nucleotide sequences will generally be appropriate for a host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used. In certain aspects, this disclosure relates to an expression vector comprising a nucleotide sequence encoding an MTM1 polypeptide and operably linked to at least one regulatory sequence. Regulatory sequences are art-recognized and are selected to direct expression of the encoded polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

This disclosure also pertains to a host cell transfected with a recombinant gene which encodes an MTM1 polypeptide, an internalizing moiety, or a chimeric polypeptide for use in the methods of the disclosure. The host cell may be any prokaryotic or eukaryotic cell. For example, an MTM1 polypeptide or a chimeric polypeptide may be expressed in bacterial cells such as E. coli, insect cells (e.g., using a baculovirus expression system), yeast, or mammalian cells. Other suitable host cells are known to those skilled in the art.

The present disclosure further pertains to methods of producing an MTM1 polypeptide, an internalizing moiety, and/or a chimeric polypeptide for use in the methods of the disclosure. For example, a host cell transfected with an expression vector encoding an MTM1 polypeptide, an internalizing moiety, or a chimeric polypeptide can be cultured under appropriate conditions to allow expression of the polypeptide to occur. The polypeptide may be secreted and isolated from a mixture of cells and medium containing the polypeptides. Alternatively, the polypeptides may be retained in the cytoplasm or in a membrane fraction and the cells harvested, lysed and the protein isolated. A cell culture includes host cells, media and other byproducts. Suitable media for cell culture are well known in the art. The polypeptides can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptides (e.g., an MTM1 polypeptide). In a preferred embodiment, the polypeptide is a fusion protein, and may optionally contain a domain which facilitates its purification.

A recombinant MTM1 nucleic acid can be produced by ligating the cloned gene, or a portion thereof, into a vector suitable for expression in either prokaryotic cells, eukaryotic cells (yeast, avian, insect or mammalian), or both. Expression vehicles for production of a recombinant polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli. The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the B-gal containing pBlueBac III).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

It should be understood that chimeric polypeptides can be made in numerous ways. For example, an MTM1 polypeptide and an internalizing moiety can be made separately, such as recombinantly produced in two separate cell cultures from nucleic acid constructs encoding their respective proteins. Once made, the proteins can be chemically conjugated directly or via a linker. By way of another example, the chimeric polypeptide can be made as an inframe fusion in which the entire chimeric polypeptide, optionally including one or more linkers, and optionally including one or more epitope tags, is made from a nucleic acid construct that includes nucleotide sequence encoding both the MTM1 polypeptide and the internalizing moiety.

### V. Methods of Treatment

The present disclosure provides certain methods of increasing muscle contractility in a subject having myotubular myopathy. Particularly, the present disclosure provides methods of increasing muscle contractility following administering low doses of chimeric polypeptide to particular patient populations (e.g., patients receiving a first dose at a particular age), as well as methods in which levels of improvement are attained following a relatively small number of doses—even at a low dosage form. These methods involve administering to an individual in need thereof a therapeutically effective amount of a chimeric polypeptide as described above. Specifically, the method comprises administering a chimeric polypeptide comprising (a) a myotubularin (MTM1) polypeptide and (b) an internalizing moiety. These methods are particularly aimed at therapeutic treatments of animals, and more particularly, humans.

In some embodiments, the present disclosure provides any of the chimeric polypeptides disclosed herein for use in increasing muscle contractility in a subject having myotubular myopathy. In some embodiments, the chimeric polypeptide comprises (a) a myotubularin (MTM1) polypeptide and (b) an internalizing moiety.

The chimeric polypeptide may be administered to a subject by any one of several different routes of administration. In some embodiments, the chimeric polypeptide is administered to a subject systemically. In some embodiments, the chimeric polypeptide is administered to a subject parenterally. In some embodiments, the chimeric polypeptide is administered to a subject intravenously, intramuscularly or subcutaneously. Intravenous delivery of recombinant MTM1 may provide the greatest flexibility in dosing with the fewest logistical barriers to development. For example, dosing of intravenous MTM1 can be titrated to effect, or withdrawn if a particular patient experiences a side effect. In some embodiments, the chimeric polypeptide is administered at one site of a subject's body, and the increased muscle contractility is observed at a different site of the subject's body (e.g, systemic effects are observed following intramuscular delivery).

MTM 1 is a cytoplasmic enzyme and possesses no inherent muscle internalizing moiety, therefore MTM1 may be conjugated to a cell permeable protein to traverse the skeletal muscle sarcolemma and reach the appropriate cytoplasmic compartments.

Additionally, hMTM1 maintains the ability to localize to early endosomes and immunoprecipitate accessory proteins such as Vps15 and Vps34 following genetic conjugation to 6-His and GST purification tags (Taylor G S et al., Proc Natl Acad Sci USA. 2000 Aug. 1; 97(16):8910-5; Cao C et al., Traffic 2007; 8: 1052-1067; Kim S A et al., J. Biol. Chem., Vol. 277, Issue 6, 4526-4531, Feb. 8, 2002), Green and Red Fluorescent Proteins (Cao C et al., Traffic 2007; 8: 1052-1067; Chaussade C et al., Molecular Endocrinology 17 (12): 2448-2460 2003; Robinson F L et al., Trends in Cell Biology, 2006, 16(8): 403-412), and flag epitope tagging (Cao C et al., Traffic 2007; 8: 1052-1067; Kim S A et al., J. Biol. Chem., Vol. 277, Issue 6, 4526-4531, Feb. 8, 2002). Therefore, chemical and genetic conjugates of 3E10 and hMTM1 will retain the ability to penetrate cells, cleave PIP3 to PI, and associate with endosomal proteins.

The terms “treatment”, “treating”, and the like are used herein to generally mean increasing the muscle contractility in a subject having MTM. “Treatment”, as used herein, may refer to an improvement in any of the following muscle weakness symptoms associated with MTM or combination thereof: respiratory insufficiency (partially or completely), poor muscle tone, drooping eyelids, poor strength in proximal muscles, poor strength in distal muscles, facial weakness with or without eye muscle weakness, abnormal curvature of the spine, joint deformities, and weakness in the muscles that control eye movement (ophthalmoplegia). Improvements in any of these conditions can be readily assessed according to standard methods and techniques known in the art. The population of subjects treated by the method or chimeric polypeptides described herein includes subjects suffering from the undesirable condition or disease, as well as subjects at risk for development of the condition or disease.

Following the administration to an MTM subject a chimeric polypeptide as described herein, an initial response is achieved. An “initial response” may refer to an increase in muscle contractility in an MTM subject by at least 50%, 75%, 100%, 150%, 200%, 250%, 300%, 350%, 400%, 450%, 500%, 550%, 600%, 650%, 700%, 750%, 800%, 850%, 900%, 1000%, 1100%, 1200%, 1300%, 1400%, 1500%, 1600%, 1700%, 1800%, 1900% or 2000% relative to that observed prior to initiation of treatment with the chimeric polypeptide.

An “initial response” may also refer to an increase in muscle contractility in an MTM subject such that the muscle contractility in the subject following the administration of the chimeric polypeptides described herein is at least 5%, 10%, 15%, 20%, 25%, 30%, 35%, 40%, 45%, 50%, 55%, 60%, 65%, 70%, 75%, 80%, 85%, 90%, 95%, or 100% of the muscle contractility in a healthy control subject. In some embodiments, the subject, prior to the administration of the chimeric polypeptides described herein, has muscle contractility that is less than 90%, 85%, 80%, 75%, 70%, 65%, 60%, 55%, 50%, 45%, 40%, 35%, 30%, 25%, 20%, 15%, 10%, 5%, 4%, 3%, 2% or 1% of the muscle contractility in a healthy control subject.

In some embodiments, the methods disclosed herein comprise administering one or more additional doses of chimeric polypeptide after achieving an initial response. In some embodiments, a “subsequent response” is achieved following the administration of one or more additional doses of the chimeric polypeptides after achieving an initial response in an MTM subject. A “subsequent response” may be the maintenance of the initial response, an improvement upon the initial response (e.g., a further increase in muscle contractility as compared to the muscle contractility level achieved in the initial response), or a decrease in muscle contractility as compared to the muscle contractility achieved in the initial response. The type of subsequent response will depend on several factors, e.g., the health and age of the subject before and during treatments. In some embodiments, the administration of one or more additional doses substantially maintains the initial response. In some embodiments, the administration of one or more additional doses provides further improvement relative to the initial response.

In some embodiments, an initial response is achieved in an MTM subject following the administration of less than or equal to 20, 19, 18, 17, 16, 15, 14, 13, 12, 11, 10, 9, 8, 7, 6, 5, 4, 3, 2, 1 doses of a chimeric polypeptide as described herein. In some embodiments, an initial response is achieved in an MTM subject following the administration of less than or equal to 4-20 doses of the chimeric polypeptide.

“Muscle Contractility”, as used herein, refers to muscle contraction that produces a measurable force and duration of power. Muscle contractility may be measured in an MTM subject by use of any number of methods well known to one of ordinary skill in the art. For example, muscle contractility may be measured by ultrasound imaging, in vivo myography, Myocyte Calcium Photometry and Contractility Systems (IonOptix), Time-Resolved Diffusing-Wave Spectroscopy, and/or luminescence resonance energy transfer, (Hodges, 2003, Muscle Nerve. 27(6):682-92; Belau et al., Apr. 11, 2010, Biomedical Optics (BIOMED), Sunday Poster Session (BSuD); Rahe-Meyer, 2007, BioMedical Engineering OnLine 2007, 6:1). In some embodiments, muscle contractility is measured in vivo by a non-invasive procedure. In other embodiments, muscle contractility is measured in vitro in a muscle biopsy sample taken from an MTM subject and/or healthy control subject. Comparisons may be made to results obtained in the same subject prior to or at an earlier stage of treatment and/or to a healthy control subject.

Muscle contractility may also be measured in an MTM subject by measuring muscle strength. Muscle Strength may be measured by using any number of methods well known to one of ordinary skill in the art. For example, muscle strength may be measured by respiratory strength tests, reflex testing, ambulatory testing, weight lifting testing, strength resistance testing, electromyography, mechanomyography, phonomyography, and/or the use of a class 1 transducer (e.g., a cantilever-baced sensor), a class 2 transducer (e.g., an optical trap), and/or a class 3 transducer (e.g., a piezo force transducer) (Hemmerling, et al., 2004, Anesthesia and Analgesia, 98(2): 377-381). In some embodiments, muscle strength is measured in vivo by a non-invasive procedure. In other embodiments, muscle strength is measured in vitro in a muscle biopsy sample taken from an MTM subject and/or healthy control subject. Comparisons may be made to results obtained in the same subject prior to or at an earlier stage of treatment and/or to a healthy control subject.

MTM subjects, or healthy control subjects, may be assessed before and after a chimeric polypeptide treatment by using any one of, or combination of, numerous different standards employed by a person having ordinary skill in the art. For example, an MTM subject, or a healthy control subject, may be assessed on a whole body level by using a scale/standard such as the AIMS (Alberta Infant Motor Scale) improvement score, the Apgar scale, the PES (perinatal evaluation score) scale, TIMP (Test of Infant Motor Performance), ENNAS (Einstein Neonatal Neurobehavioral Assessment Scale), PEDI (Pediatric Evaluation of Disability Index), GMsA (General Movements Assessment), CHOP Childrens Hospitasl of Philadelphia scale), GMFM (Gross Motor Function Measurement), Hammersmith Functional Motor Scale), BSIDT (Bagley Scales of Infant and Toddler Development, a comparison to a healthy subject or a responder analysis (i.e., comparison to baseline). These are several representative scales/standards that may be used for assessing the efficacy of chimeric polypeptide treatment to an MTM subject or a healthy control subject, and the skilled worker can easily envision further scales/standards for assessing the efficacy of the methods of the present disclosure.

By the term “therapeutically effective dose” or “effective amount” is meant a dose that produces the desired effect for which it is administered. The exact dose will depend on the purpose of the treatment, and will be ascertainable by one skilled in the art using known techniques (see, e.g., Lloyd (1999) The Art, Science and Technology of Pharmaceutical Compounding).

In some embodiments, the first dose of a chimeric polypeptide is administered to an MTM subject after the subject is 5, 6, 7, 8, 9, 10, 11, 12, 13, 14, 15, 16, 17 or 18 years of age. In other embodiments, the first dose of a chimeric polypeptide is administered to an MTM subject before the subject is 5 years, 4 years, 3 years, 2 years, 1 year, 11 months, 10 months, 9 months, 8 months, 7 months, 6 months, 5 months, 4 months, 3 months, 2 months, 1 month, 3 weeks, 2 weeks, 1 week, 6 days, 5 days, 4 days, 3 days, 2 days, or 1 day of age.

Methods of treating include administering to an MTM subject the chimeric polypeptides according to a dosing regimen. In some embodiments, the dosing regimen involves the administration of the chimeric polypeptides according to a single dose or multiple doses. Multiple doses include administering the chimeric polypeptide at specified intervals, such as daily, weekly, twice monthly, monthly, etc. In some embodiments, the chimeric polypeptide is administered to the MTM subject at least once over a two week period, at least once over a one week period, at least twice over a one week period, or at least once a day.

Methods of treating include administering at least 1, 2, 3, 4, 5, 6, 7, 8, 9, 10, 11, 12, 13, 14, 15, 16, 17, 18, 19, 20 doses to the MTM subject before an initial response is achieved. In some embodiments, the methods described herein comprise administering one or more additional doses of chimeric polypeptide even after achieving an increase in muscle contractility. In some embodiments, the methods described herein comprise administering the chimeric polypeptide to the subject throughout the lifetime of the subject. In other embodiments, the methods described herein comprise administering the chimeric polypeptide to the subject until the subject is asymptomatic for myotubular myopathy.

In some embodiments, the methods or chimeric polypeptides for use in increasing muscle contractility, as disclosed herein, increase muscle contractility in at least a subset of muscles in an MTM subject, and the increase in muscle contractility is effective to improve respiratory function in the subject. In some embodiments, the methods or chimeric polypeptides disclosed herein increase muscle contractility in at least a subset of muscles in an MTM subject, and the increase in muscle contractility is effective to increase mobility in said subject. In some embodiments, the methods or chimeric polypeptides disclosed herein decrease the subject's reliance on a respirator.

In preferred embodiments, the muscle with increased contractility following the administration of the chimeric polypeptides described herein is skeletal muscle. In some embodiments, the skeletal muscle comprises Type I and/or Type II muscle fibers. In some embodiments, the Type II muscle fibers are Type IIa, Type IIb and/or Type IIx fibers. In some embodiments, the skeletal muscles are diaphragm muscles, facial muscles, paraspinal muscles, erector spinae muscles, lower limb muscles and/or upper limb muscles. In some embodiments, the facial muscles are eyelid, jaw, tongue, lips, mouth and/or throat muscles.

### VI. Methods of Administration

Various delivery systems are known and can be used to administer the chimeric polypeptides of the disclosure, e.g., various formulations, encapsulation in liposomes, microparticles, microcapsules, recombinant cells capable of expressing the compound, receptor-mediated endocytosis (see, e.g., Wu and Wu, 1987, J. Biol. Chem. 262:4429-4432). Methods of introduction can be enteral or parenteral, including but not limited to, intradermal, transdermal, intramuscular, intraperitoneal, intravenous, subcutaneous, pulmonary, intranasal, intraocular, epidural, and oral routes. In particular embodiments, parenteral introduction includes intramuscular, subcutaneous, intravenous, intravascular, and intrapericardial administration.

The present disclosure provides systemic delivery of one or more doses of a chimeric polypeptide of the disclosure. Systemic delivery includes, for example, subcutaneous, intravenous, or intramuscular. In fact, the results described herein demonstrate that, following intramuscular delivery of chimeric polypeptide, therapeutic efficacy is observed in other muscles (e.g., not limited to the injected muscle). This is not the case following intramuscular delivery of all agents and indicates that the chimeric polypeptide is available systemically following intramuscular administration. However, in the context of the present disclosure systemic administration includes, in certain embodiments, intramuscular delivery. In other embodiments, the systemic administration is via another route and is not intramuscular delivery.

The chimeric polypeptides may be administered by any convenient route, for example, by infusion or bolus injection.

In certain embodiments, the chimeric polypeptides are administered by intravenous infusion. In certain embodiments, the chimeric polypeptides are infused over a period of at least 10, at least 15, at least 20, or at least 30 minutes. In other embodiments, the chimeric polypeptides are infused over a period of at least 60, 90, or 120 minutes. Regardless of the infusion period, the disclosure contemplates that each infusion is part of an overall treatment plan where chimeric polypeptide is administered according to a regular schedule (e.g., weekly, monthly, etc.).

### VII. Pharmaceutical Compositions

In certain embodiments of the methods of the present disclosure, the subject chimeric polypeptides are formulated with a pharmaceutically acceptable carrier. One or more chimeric polypeptides can be administered alone or as a component of a pharmaceutical formulation (composition).

The present disclosure also provides for pharmaceutical preparations comprising any of the chimeric polypeptides disclosed herein for use in increasing muscle contractility in a subject having myotubular myopathy.

The chimeric polypeptides may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the chimeric polypeptides include those suitable for oral, nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the compound which produces a therapeutic effect.

In certain embodiments, methods of preparing these formulations or compositions include combining the therapeutic agent and a carrier and, optionally, one or more accessory ingredients. In general, the formulations can be prepared with a liquid carrier, or a finely divided solid carrier, or both, and then, if necessary, shaping the product.

Pharmaceutical compositions suitable for parenteral administration may comprise one or more chimeric polypeptides in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

In certain embodiments, the chimeric polypeptides for use in the methods of the present disclosure are formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The amount of the chimeric polypeptides for use in the methods of the present disclosure which will be effective in the treatment of myotubular myopathy can be determined by standard clinical techniques. As shown herein, a low dosage form, administered in just a few doses, is efficacious in improving muscle contractility even in severely affected mice. Specifically, administration to mice of less than 5 ug/dose (e.g., 2 ug/dose; a dosage of approximately 0.1 mg/kg) administered in a 0.1 mg/ml formulation was efficacious after just four doses.

Effective doses may be extrapolated from dose-response curves derived from in vitro or animal model test systems. In the context of the present disclosure, the effective dose is adjusted to provide an initial response, as described herein, achieved in fewer than 20 doses (e.g., less than 15, less than 10, less than 8, less than 5, etc.). For example, the effective dose is adjusted to provide an initial response in terms of an increase in muscle contractility achieved in fewer than 20 doses. In certain embodiments, the initial response is achieved in less than or equal to 10, 9, 8, 7, 6, 5, 4, 3, or less than or equal to 2 doses.

In certain embodiments, chimeric polypeptides and compositions discussed herein, including pharmaceutical preparations, are non-pyrogenic. In other words, in certain embodiments, the compositions are substantially pyrogen free. In one embodiment the formulations of the disclosure are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in relatively large dosages and/or over an extended period of time (e.g., such as for the patient's entire life), even small amounts of harmful and dangerous endotoxin could be dangerous. In certain specific embodiments, the endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

In another aspect, the disclosure provides a pharmaceutical composition comprising a chimeric polypeptide of the disclosure formulated in a pharmaceutically acceptable carrier, such as with one or more pharmaceutically acceptable excipients. In certain embodiments, the pharmaceutical composition comprises a polypeptide comprising the amino acid sequence set forth in SEQ ID NO: 11 or SEQ ID NO: 18, in the presence or absence of one or both epitope tags.

The foregoing applies to any of the methods described herein. The disclosure specifically contemplates any combination of the features of such chimeric polypeptides and compositions for use in the methods of the present disclosure (alone or in combination) with the features described for the various pharmaceutical compositions and route of administration described in this section.

### VII. Animal Models of MTM

The methods, chimeric polypeptides or pharmaceutical preparations of the disclosure may be tested in any one of several animal models in order to optimize dosing or the generation of formulations.

Mice possessing a targeted inactivation of the MTM1 gene (MTM1 KO) are born at a submendellian distribution but otherwise appear normal. However, within the first weeks of life MTM1 KO mice begin to lose muscle mass that rapidly progresses to respiratory collapse and death at a median age of 7 weeks (14 weeks maximum). Myofibers of MTM KO mice appear hypotrophic and vacuolated with centrally located nuclei surrounded by mitochondria and glycogen, yet there is very little sarcolemma damage and no evidence of apoptosis or inflammation. Ultrastructurally, MTM1 protein appears at submembranous and vesicles of the cytoplasm; and the triads of the T-tubule system of skeletal muscle (Bello A B et al., Proc Natl Acad Sci USA. 2002 Nov. 12; 99(23):15060-5). Since the deficiency of MTM1 in skeletal muscle solely accounts for the phenotype in MTM1 KO mice, the constructs disclosed herein may be assessed for therapeutic efficacy using the MTM1 KO mouse model. Further, mice possessing a targeted partial inactivation of the MTM1 gene can also serve as a suitable model system for the present disclosure. Such mouse models are known in the art. For example, in MTM1δ4 mice, exon 4 is replaced by a loxP site and the Cre allele is absent (Buj-Bello et al., 2002, PNAS 99(23):15060-15065).

Mice possessing an R69C mutation model human cases of MTM in which a recurrent R69C missense mutation occurs. These mice, like the human cases they model, are associated with variable degrees of altered splicing that may lead to loss of exon 4 in the Mtm1 gene. The Mtm1p.R69C mouse models display stable weakness starting at 2-3 months of life and a mean lifespan of 60 weeks. (Piersen et al., 2012, Hum Mol Genet, 21: 811-825).

Accordingly, in certain embodiments, the present disclosure contemplates methods of surveying increases in muscle contractility using the chimeric polypeptides disclosed herein in a mouse model of MTM. Studies in MTM1 deficient mice demonstrate the marked phenotypic differences between wild-type and MTM1 deficient mice (see, e.g., Buj-Bello et al., 2002, PNAS 99(23):15060-15065). For example, a clear divergence in weight gain between normal and MTM1 deficient mice can be seen at ˜3 weeks of age. (Bello A B et al., Proc Natl Acad Sci USA. 2002 Nov. 12; 99(23):15060-5) Also, hanging assessment tests indicate a dramatic difference in the hanging performance between MTM1 deficient mice and normal mice. Additionally, MTM1 deficient mice demonstrate a significant deterioration in grip strength (e.g., forelimb grip) as compared to normal mice. Further, compared to normal mice which manifest almost no foot dragging, MTM 1 deficient mice demonstrate increased foot dragging as determined by gait analysis. Detailed protocols for evaluating the effect of chimeric polypeptides comprising MTM1 in this animal model are described herein (Example 4).

As such, upon administration (e.g., intravenously or intramuscularly) to the MTM1 deficient mice, the ability of the chimeric polypeptides disclosed herein to increase muscle contractility in MTM1 deficient mice may be assessed using any one of, or combination of, the muscle contractility (or muscle strength) assays known to the skilled worker (e.g., those assays described herein).

The above mouse models provide a suitable animal model system for assessing the activity and effectiveness of the chimeric polypeptides as a means for increasing muscle contractility in a subject. Further, these models correlate strongly with MTM, and provide an appropriate model for MTM. Activity of the polypeptide can be assessed in these mouse models, and the results compared to that observed in wildtype control animals and animals not treated with the chimeric polypeptides. The results can be evaluated by examining the mice, and by using any of the muscle contractility (or muscle strength) assays known to the skilled worker (e.g., including those assays described herein). Treated mice can also be assessed using standard tests used to evaluate muscle strength in mice, e.g., by performing hanging grip tests, rotarod endurance performance tests, and/or treadmill tests. Moreover, treated mice can be observed to evaluate differences in weight, behavior, mobility, etc. Similarly, the efficacy of the subject chimeric polypeptides can be evaluated using cells in culture, for example, cells prepared from the mutant mice.

In other embodiments, a large animal model can also be used to assess the activity and effectiveness of the subject chimeric polypeptides. By way of example, a dog model may be a particularly useful system for studying MTM. The affected dog carries a deficient MTM1 gene and, therefore, the studies described herein for a mouse model similarly apply to a dog model. The evaluation dose of 3E10 chemically or genetically conjugated to hMTM1 delivered to MTM1 deficient dogs will be determined empirically.

In other embodiments, a zebrafish model can be used to assess the activity and effectiveness of the subject chimeric polypeptides. A zebrafish model of MTM has been generated by utilizing a morpholino knockdown system that reduces MTM1 expression in these animals. The MTM zebrafish model displays significantly impaired motor function, myofiber pathology and depressed muscle contractility (Dowling et al., 2009, PLoS Genetics, 5(2): e1000372). In view of these phenotypes, the zebrafish may be a useful model on which to perform the methods of the present disclosure.

### Exemplification

The disclosure now being generally described, it will be more readily understood by reference to the following examples, which are included merely for purposes of illustration of certain aspects and embodiments of the present disclosure, and are not intended to limit the disclosure.

### EXAMPLE 1

### Production and Characterization of 3E10Fv-MTM1

An exemplary chimeric polypeptide was made as a fusion protein. This chimeric polypeptide has an antibody fragment portion located N-terminal to human myotubularin. Specifically, the antibody fragment portion is an scFv comprising an exemplary 3E10 light chain variable domain (SEQ ID NO: 4) interconnected via a linker to an exemplary 3E10 heavy chain variable domain (SEQ ID NO: 2), and this antibody fragment is N-terminal to human myotubularin (SEQ ID NO: 1). The scFv comprises the 6 CDRs set forth in SEQ ID NOs 12-17. Thus, from N-terminus to C-terminus, the polypeptide comprises: exemplary 3E10 VL-linker-exemplary 3E10 VH-linker-myotubularin. Optionally, there may be one or more epitope tags interspersed or present in this construct. The specific chimeric polypeptide used for the studies summarized below is now described in more detail.

A gene containing an N-terminal GST tag with a Thrombin cleavage site and C-terminal Myc6× His tag (GST-3E10Fv-MTM1-myc6His) was codon-optimized for E. coli expression and was synthesized de novo (Millipore; Temecula, Calif.). The GST-3E10Fv-MTM1-myc6His gene (encoding a protein having the sequence of SEQ ID NO: 18) was cloned into the pGEX-2T GST expression vector and transformed in an E. coli strain. Following expression optimization and purification with a Glutathione Sepharose 4B column, the GST tag was removed by Thrombin Cleavage. Purified 3E10Fv-MTM1-myc6His protein, (also referred to herein as 3E10Fv-MTM1), was examined for purity by SDS-PAGE, and identity was confirmed by positive reactivity with an anti-HIS mouse monoclonal antibody [#05-949;Millipore (Upstate), USA] by Western blot and material was dialyzed with TBS buffer. 3E10Fv-alone was produced as a control protein in E. Coli utilizing similar expression methods and was stored in PBS buffer following purification (Millipore, USA). As noted above, the 3E10Fv used has the 6 CDRs set forth in SEQ ID NOs: 12-17. The antibody fragment in the specific construct set forth in SEQ ID NO: 18 is a murine antibody fragment. However, humanized antibodies and antibody fragments comprising the 6 CDRs set forth in SEQ ID NOs: 12-17 are also contemplated and may similarly be used (e.g., chimeric polypeptides in which the antibody fragment portion of, for example, SEQ ID NO: 18, is humanized).

To determine whether the 3E10Fv-MTM1 chimeric polypeptide retained the phosphoinositide phosphatase function of myotubularin, the 3E10Fv-MTM1 chimeric polypeptide was tested in a phosphatase assay. 3E10Fv-MTM1-catalyzed dephosphorylation of phosphatidylinositol 3,5-bisphophate (Ptdlns(3,5)P₂) was carried out in a 20 μl reaction mixture pH 6.5 consisting of a 1× dilution of 10× reaction buffer [0.5 M Citric Acid and 0.5 M NaCl] with 50 μM Ptdlns(3,5)P₂ (Echelon; catalog #p-3508), 200 μM biotin-labeled phosphatidylserine (PS) (Echelon; catalog #L-31B16), and 0.05 μg of the 3E10Fv-MTM1 enzyme fusion. Briefly, 3E10Fv-MTM1 fusion protein or vehicle was added to reaction buffer for two minutes at 37° C. before addition of substrate and subsequent incubation time of 20 minutes. The reactions were terminated by the addition of 15 μl of 100 mM N-ethyl malemide (NEM) in DMSO and left at room temperature for three minutes before placing on ice and centrifuging at 14,000 rpm for 15 minutes. A portion of the supernatant (20 μl) was treated with 80 μl of Malachite green solution and assayed alongside a standard curve of inorganic phosphate (Pᵢ) solutions according to manufacturer's suggestions (Echelon, #K-1500). Color was allowed to develop for 20 minutes at room temperature before absorbance at 595 nm was measured, and phosphate release generated by 3E10Fv-MTM1 incubation was quantified in comparison to (Pᵢ) standards. The phosphatase assay was measured in triplicate in two experiments (i.e., a total of six reactions).

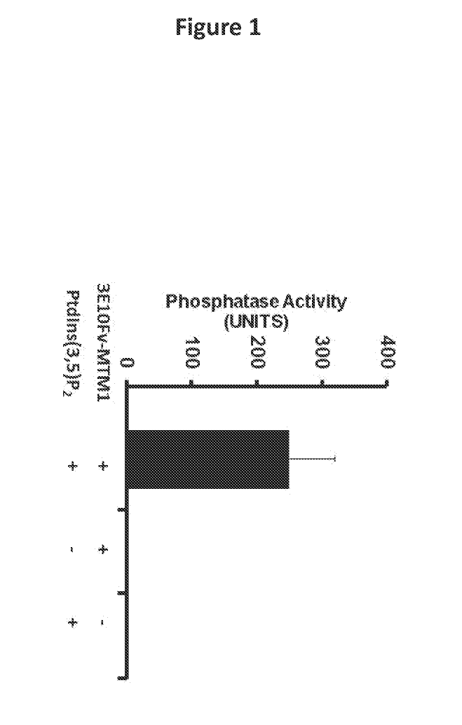

FIG. 1 illustrates the average results from the phosphatase assay experiments. The results demonstrate that the 3E10Fv-MTM1 chimeric protein was associated with a mean Pᵢ generation of 249.83+/−70.07 pmoles per 20 μl reaction over the course of 20 minutes, whereas reactions deficient in chimeric protein and/or substrate showed no detectable Pᵢ release. Compared to previously published data on MTM1-alone phosphatase activity, the 3E10Fv-MTM1 protein showed comparable calculated activity, especially, when considering the proportional molecular weight of MTM1 as a percentage (˜70%) of the entire 3E10Fv-MTM1 fusion protein. (Schaletzky, J., et al. 2003, Current Biology: CB 13, 504-509; Tronchere, H., et al., 2004, J Biol Chem 279, 7304-7312.)

### EXAMPLE 2

### Administration of 3E10Fv-MTM1 to an Mtm1δ4 Animal Model

To address the question of whether therapeutic delivery of a low dosage of myotubularin would improve muscle function in an animal model having a more severe form of MTM, Mtm1δ4 mice were given intramuscular injections of 3E10Fv-MTM1. Intramuscular injection was employed so that both local effects in the injected TA muscle and potential systemic effects of the disseminated 3E10Fv-MTM1 conjugate could be investigated.

Male Mtm1δ4 mice (n=5) were injected intramuscularly into the right tibialis anterior (“TA”) muscle with 20 ul of 0.1 mg/mL 3E10Fv-MTM1 starting at 28 days of life. This dosage was chosen based on the estimated efficacy of 3E10-delivery compared with the dose and dose interval of other non-targeted enzyme replacement therapies. Thus, this represents a low dose of therapeutic agent. Control male Mtm1δ4 mice were injected with equivalent volumes of tris buffered saline (n=5) or unconjugated 3E10Fv-alone (n=5) using the same dosing schedule. All animals were injected twice weekly until 42 days of life, with a total of 4 doses in the two-week period. At 42 days of life, animals were euthanized, photographed, and muscle contractility was assessed ex vivo in muscle samples taken directly from the mice from the different treatment groups. In addition, the tibialis anterior, soleus, quadriceps, gastrocnemius, triceps, and diaphragm muscles were carefully dissected, weighed, frozen using isopentane, and stored at −80° C. for subsequent histological studies.

Muscle contractility was tested using a Graz bath procedure. Briefly, the right EDL (extensor digitorum longus) muscle from Mtm1δ4 mice from the different treatment groups were carefully dissected immediately following euthanasia and external photography of the animal, and 4-0 sutures were tied around the proximal and distal tendons. The muscles were placed into heated (30° C.), oxygenated (95% O₂, 5% CO₂) Krebs Henseleit buffer (pH 7.4; Sigma) containing 0.2 grams of calcium chloride and 1.8 grams of sodium bicarbonate added per liter. Muscles were mounted onto a 4-channel Graz tissue bath apparatus (Harvard Apparatus) connected to a Powerlab data collection system using Chart 5 software. Muscles were stimulated by square pulses of 0.2 ms duration at a voltage and muscle length (L0) to elicit maximal isometric twitch force. The output stimulus was derived from a Hugo Sachs Elektronik type 215E13 Voltage Pulse Generator (Harvard Apparatus) triggered at the desired frequency. Based on preliminary studies, the maximal isometric twitch response was elicited at both ages tested with a resting tension of 1.0 g and a voltage set at 10 V. Each muscle was pre-tensioned to a force of 1 gram. After an initial equilibration and test stimulation period, the muscle was subjected to a tension-frequency protocol at electrical stimulation frequencies of 1, 10, 20, 30, 50, 80, 100, 120, 150, and 180 Hz, each spaced 1 minute apart. The pre-tension force was reset to 1.0 g before each stimulus. After completion of the tension-frequency protocol, the length of these muscles at a pre-tension of 1.0 g was then measured, and the muscle tissue between the sutures was weighed after trimming off the suture and excess tissue. Estimated cross-sectional area (CSA) was calculated by dividing the mass of the muscle (g) by the product of its length (cm) and the density of muscle (1.06 g/cm3) and expressed as square millimeters. Muscle output was expressed as stress (g/mm²) determined by dividing the tension (g) by muscle CSA.

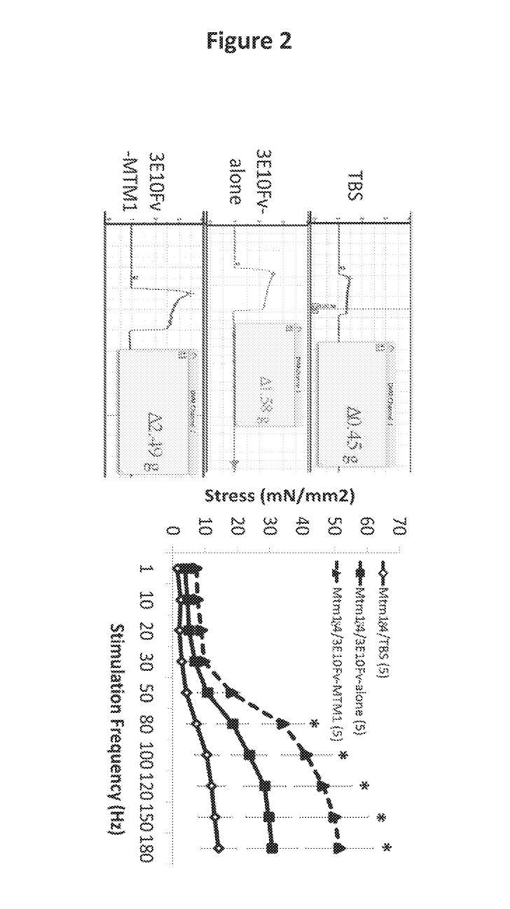

Surprisingly, Mtm1δ4 mice treated with just four doses of therapeutic construct delivered at a low dosage of just 0.1 mg/kg had improved muscle contractility, and systemic effects were observed (e.g., efficacy observed in muscles that were not injected). Functional testing of isolated EDL muscles from the treated Mtm1δ4 mice demonstrated an improvement in strength. Mean tetanic normalized stress of 3E10Fv-MTM1 treated EDLs was 5.10 mN/mm2 (range=3.05-9.25 mN/mm2), or 386% of mean saline-injected muscle values, and 18% of untreated age-matched WT values (FIG. 2).

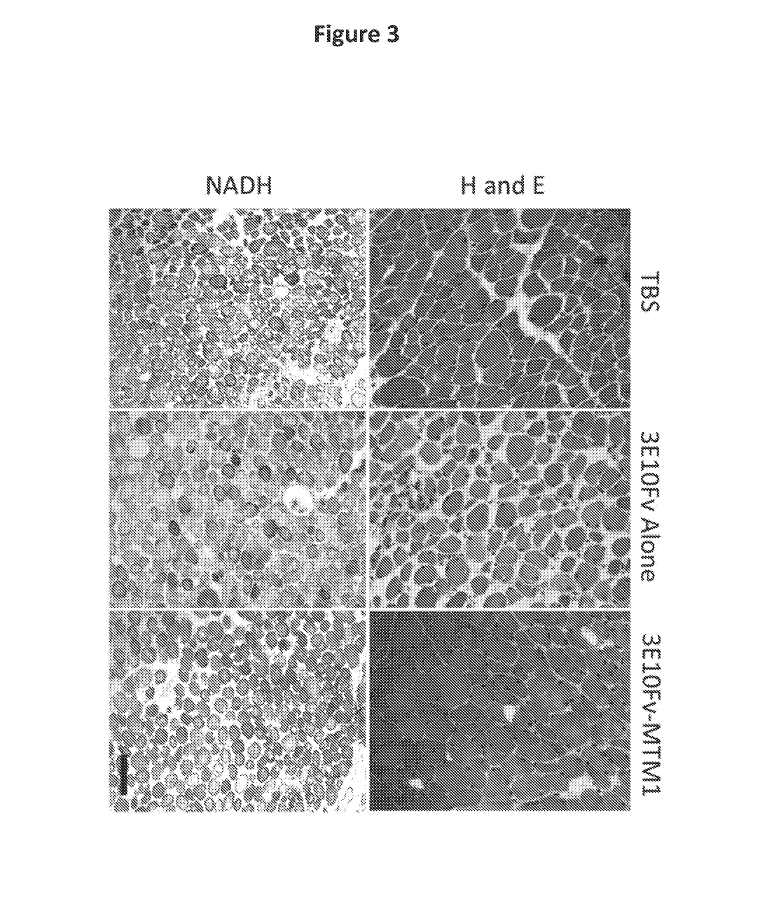

Muscle samples from the different treatment groups of Mtm1δ4 mice were also taken and assessed for histological abnormalities. Cross sections (8 μm) of isopentane-frozen quadriceps muscle were taken midway down the length of the muscle and stained with hematoxylin and eosin (H and E) (FIG. 3). For NADH staining, frozen sections were incubated with nitro-blue tetrazolium (1 mg/ml, Sigma) and beta-nicotinamide adenine dinucleotide (0.4 mg/ml, Sigma) in 50 mM Tris-HCl, pH 7.3, at 25° C. for 30 minutes. Light microscopic images were captured using an Olympus DP72 camera and cellSens Standard software (Olympus, Center Valley, Pa.). The number of centrally-nucleated fibers and necklace fibers were quantified by evaluating non-overlapping images of H and E or NADH stains, respectively, and manually counting the number of fibers containing these structures.

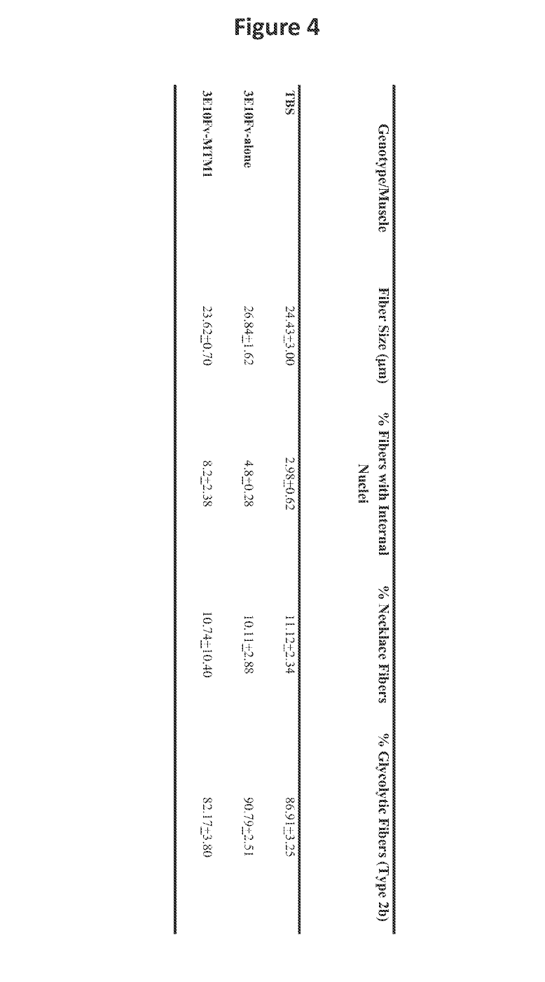

The locally injected TA muscles did not show differences in disease pathology at the light microscopic level (FIGS. 3 and 4). The EDL of Mtm1δ4 mice from the different treatment groups displayed similar pathology, i.e., reduced myofiber size, increased numbers of centrally-nucleated fibers, and increased numbers of necklace fibers (FIG. 4). In other words, despite the improvement in muscle contractility observed following just four low dose treatments, increases in myofiber size were not observed.

Surprisingly, the control condition, unconjugated 3E10Fv-alone, also moderately improved contractile function in EDL muscles of Mtm1δ4 mice (FIG. 4); although not at the same levels as the chimeric polypeptide. EDL muscles from animals injected with 3E10Fv-alone produced mean tetanic normalized stress values of 29.6 mN/mm2, which corresponds to 229% of TBS-injected values and 59% of 3E10Fv-MTM1-injected values (FIG. 2). Injection of 3E10Fv-alone did not affect the pathology of the TA muscles at the light microscopic level (FIGS. 3 and 4).

In addition to improvements in muscle contractility, Mtm1δ4 mice also displayed noticeable improvements in gross muscle strength after being administered only four doses of 3E10Fv-MTM1. Specifically, Mtm1δ4 mice subjectively appeared more mobile in their cages after only four doses of this low dosage form of 3E10Fv-MTM1.

Increased muscle strength is also evaluated using strength assays well known to one of ordinary skill in the art. For example, muscle strength is evaluated in Mtm1δ4 mice from the different treatment groups by performing hanging grip tests, rotarod endurance performance tests, and/or treadmill tests. For treadmill tests, mice are placed on a treadmill with a rear electrical shock (e.g. AccuPacer Treadmill, AccuScan Instruments Inc.). The speed is increased by 2 m/min every two minutes for 30 minutes or until mouse is unable to run.

In this study, muscle contractility was evaluated in isolated muscle. However, muscle strength and/or muscle contractility can be evaluated using alternative assays in living subjects. In this way, changes of muscle strength can be assayed in the same animal prior to initiation of treatment, and compared to that observed over time once treatment has been initiated (e.g., following 2, 3, 4, 5, 6, 10, 12, 14, 16, 20, more than 20 doses, etc.). Similarly, changes of muscle strength can be assay in different muscles.

### EXAMPLE 3

### Administration of 3E10Fv-MTM1 to an MTM1 p.R69C Animal Model

To address the question of whether therapeutic delivery of low dose myotubularin would improve muscle function in an animal model having a milder form of MTM, MTM1 p.R69C mice are given intramuscular injections of 3E10Fv-MTM1. Intramuscular injection is employed so that both local effects in the injected TA muscle and potentially systemic effects of the disseminated 3E10Fv-MTM1 conjugate can be investigated. This model is also useful for evaluating dosing regimens for treating older patients (e.g., those diagnosed later or those who have survived with varying levels of disability prior to the availability of MTM1 chimeric polypeptide therapy).

Male 3E10Fv-MTM1 mice (n=5) are injected intramuscularly into the right tibialis anterior (“TA”) muscle with 20 ul of 0.1 mg/mL3E10Fv-MTM1 starting at 56 days of life. This dosage is chosen based on the estimated efficacy of 3E10-delivery compared with the dose and dose interval of other non-targeted enzyme replacement therapies. Control male 3E10Fv-MTM1 mice are injected with equivalent volumes of tris buffered saline (n=5) or unconjugated 3E10Fv-alone (n=5) using the same dosing schedule. Other controls are wildtype male mice injected with tris buffered saline (n=5), unconjugated 3E10Fv-alone (n=5) or 3E10Fv-MTM1 using the same dosing schedules. All animals are injected twice weekly until 40 weeks of life. At 40 weeks of life, animals are euthanized, photographed, and contractile function of the right EDL muscle is evaluated using, for example, a Graz bath as described above. The tibialis anterior, soleus, quadriceps, gastrocnemius, triceps, and diaphragm muscles are carefully dissected, weighed, frozen using isopentane, and stored at −80° C. for subsequent histological studies.

Increased muscle strength is also evaluated using strength assays well known to one of ordinary skill in the art. For example, muscle strength is evaluated in MTM1 p.R69C mice from the different treatment groups by performing hanging grip tests, rotarod endurance performance tests, and/or treadmill tests. For treadmill tests, mice are placed on a treadmill with a rear electrical shock (e.g. AccuPacer Treadmill, AccuScan Instruments Inc.). The speed is increased by 2 m/min every two minutes for 30 minutes or until mouse is unable to run.

However, muscle strength and/or muscle contractility can be evaluated using alternative assays in living subjects. In this way, changes of muscle strength can be assayed in the same animal prior to initiation of treatment, and compared to that observed over time once treatment has been initiated (e.g., following 2, 3, 4, 5, 6, 10, 12, 14, 16, 20, more than 20 doses, etc.). Similarly, changes of muscle strength can be assay in different muscles.

### EXAMPLE 4

### Assessment of Alternative Systemic Administration Methods in Mtm1δ4 Mice

As discussed in Example 2, intramuscular injection of Mtm1δ4 mice with 3E10Fv-MTM1 was associated with an efficacious systemic exposure to 3E10Fv-MTM1 in the treated mice. Systemic administration of low dosage 3E10Fv-MTM1 by other routes is tested in Mtm1δ4 mice to determine improved contractility following limited number of doses.

Male Mtm1δ4 mice (n=5) are injected intravenously (e.g., by tail vein) or subcutaneously (e.g. into skin between shoulder blades) with 20 ul of 0.1 mg/mL 3E10Fv-MTM1 starting at 28 days of life. Control male mice are injected with equivalent volumes of tris buffered saline (n=5) or unconjugated 3E10Fv-alone (n=5) using the same dosing schedule. Other controls are wildtype male mice injected by the same routes with tris buffered saline (n=5), unconjugated 3E10Fv-alone (n=5) or 3E10Fv-MTM1 using the same dosing schedules. All animals are injected twice weekly until 42 days of life, with a total of 4 doses in the two-week period. At 42 days of life, animals are euthanized, photographed, and muscle contractility is assessed ex vivo in muscle samples taken directly from mice from the different treatment groups. In addition, the tibialis anterior, soleus, quadriceps, gastrocnemius, triceps, and diaphragm muscles are carefully dissected, weighed, frozen using isopentane, and stored at −80° C. for subsequent histological studies. Muscle contractility, motility, muscle strength and disease pathology are then evaluated, for example, by the methods described in Example 2.

### EXAMPLE 5

### Assessment of Alternative Systemic Administration Methods in MTM1 p.R69C Mice

As discussed in Example 2, intramuscular injection of Mtm1δ4 mice with 3E10Fv-MTM1 was associated with an efficacious systemic exposure to 3E10Fv-MTM1 in the treated mice. Systemic administration of 3E10Fv-MTM1 by other routes is tested in MTM1 p.R69C mice to determine whether the same or improved contractility may be achieved if 3E10Fv-MTM1 is administered by routes other than intramuscular injection.

Male MTM1 p.R69C mice (n=5) are injected intravenously (e.g., by tail vein) or subcutaneously (e.g. into skin between shoulder blades) with 20 ul of 0.1 mg/mL 3E10Fv-MTM1 starting at 56 days of life. Control male 3E10Fv-MTM1 mice are injected with equivalent volumes of tris buffered saline (n=5) or unconjugated 3E10Fv-alone (n=5) using the same dosing schedule. Other controls are wildtype male mice injected by the same route with tris buffered saline (n=5), unconjugated 3E10Fv-alone (n=5) or 3E10Fv-MTM1 using the same dosing schedules. All animals are injected twice weekly until 40 weeks of life. At 40 weeks of life, animals are euthanized, photographed, and contractile function of the right EDL muscle is evaluated. The tibialis anterior, soleus, quadriceps, gastrocnemius, triceps, and diaphragm muscles are carefully dissected, weighed, frozen using isopentane, and stored at −80° C. for subsequent histological studies. Muscle contractility, motility, muscle strength and disease pathology are then evaluated, for example, by the methods described in Example 2.

### Statistical Analysis:

Statistical analyses were performed using Prism 5 software (GraphPad Software, San Diego, Calif.). Individual datasets were compared using ANOVA analyses and Bonferonni post-tests. Differences were considered to be statistically significant at P<0.05. All data are presented as means±Standard Error of the Mean (SEM).

### SEQUENCE INFORMATION

amino acid sequence of the human MTM1 protein (NP_000243.1)
SEQ ID NO: 1
MASASTSKYNSHSLENESIKRTSRDGVNRDLTEAVPRLPGETLITDKEVIYICPFNGPIKGRVYI
TNYRLYLRSLETDSSLILDVPLGVISRIEKMGGATSRGENSYGLDITCKDMRNLRFALKQEGHSR
RDMFEILTRYAFPLAHSLPLFAFLNEEKFNVDGWTVYNPVEEYRRQGLPNHHWRITFINKCYELC
DTYPALLVVPYRASDDDLRRVATFRSRNRIPVLSWIHPENKTVIVRCSQPLVGMSGKRNKDDEKY
LDVIRETNKQISKLTIYDARPSVNAVANKATGGGYESDDAYHNAELFFLDIHNIHVMRESLKKVK
DIVYPNVEESHWLSSLESTHWLEHIKLVLTGAIQVADKVSSGKSSVLVHCSDGWDRTAQLTSLAM
LMLDSFYRSIEGFEILVQKEWISFGHKFASRIGHGDKNHTDADRSPIFLQFIDCVWQMSKQFPTA
FEFNEQFLIIILDHLYSCRFGTFLFNCESARERQKVTERTVSLWSLINSNKEKFKNPFYTKEINR
VLYPVASMRHLELWVNYYIRWNPRIKQQQPNPVEQRYMELLALRDEYIKRLEELQLANSAKLSDP
PTSPSSPSQMMPHVQTHF
exemplary 3E10 Variable heavy chain
SEQ ID NO: 2
EVQLVESGGGLVKPGGSRKLSCAASGFTFSNYGMHWVRQAPEKGLEWVAYISSGSSTIYYADTVK
GRFTISRDNAKNTLFLQMTSLRSEDTAMYYCARRGLLLDYWGQGTTLTVSS
linker sequence “G53”
SEQ ID NO: 3
GGGGSGGGGSGGGGS
exemplary 3E10 Variable light chain
SEQ ID NO: 4
DIVLTQSPASLAVSLGQRATISCRASKSVSTSSYSYMHWYQQKPGQPPKLLIKYASYLESGVPAR
FSGSGSGTDFHLNIHPVEEEDAATYYCQHSREFPWTFGGGTKLELK
human MTM1 nucleic acid sequence (NM_000252.2)
SEQ ID NO: 5
agagggggcg gagcagggcc cggcagccga gcagcctggc aacggcggtg gcgcccggag
cccgagagtt tccaggatgg cttctgcatc aacttctaaa tataattcac actccttgga
gaatgagtct attaagagga cgtctcgaga tggagtcaat cgagatctca ctgaggctgt
tcctcgactt ccaggagaaa cactaatcac tgacaaagaa gttatttaca tatgtccttt
caatggcccc attaagggaa gagtttacat cacaaattat cgtctttatt taagaagttt
ggaaacggat tcttctctaa tacttgatgt tcctctgggt gtgatctcga gaattgaaaa
aatgggaggc gcgacaagta gaggagaaaa ttcctatggt ctagatatta cttgtaaaga
catgagaaac ctgaggttcg ctttgaaaca ggaaggccac agcagaagag atatgtttga
gatcctcacg agatacgcgt ttcccctggc tcacagtctg ccattatttg catttttaaa
tgaagaaaag tttaacgtgg atggatggac agtttacaat ccagtggaag aatacaggag
gcagggcttg cccaatcacc attggagaat aacttttatt aataagtgct atgagctctg
tgacacttac cctgctcttt tggtggttcc gtatcgtgcc tcagatgatg acctccggag
agttgcaact tttaggtccc gaaatcgaat tccagtgctg tcatggattc atccagaaaa
taagacggtc attgtgcgtt gcagtcagcc tcttgtcggt atgagtggga aacgaaataa
agatgatgag aaatatctcg atgttatcag ggagactaat aaacaaattt ctaaactcac
catttatgat gcaagaccca gcgtaaatgc agtggccaac aaggcaacag gaggaggata
tgaaagtgat gatgcatatc ataacgccga acttttcttc ttagacattc ataatattca
tgttatgcgg gaatctttaa aaaaagtgaa ggacattgtt tatcctaatg tagaagaatc
tcattggttg tccagtttgg agtctactca ttggttagaa catatcaagc tcgttttgac
aggagccatt caagtagcag acaaagtttc ttcagggaag agttcagtgc ttgtgcattg
cagtgacgga tgggacagga ctgctcagct gacatccttg gccatgctga tgttggatag
cttctatagg agcattgaag ggttcgaaat actggtacaa aaagaatgga taagttttgg
acataaattt gcatctcgaa taggtcatgg tgataaaaac cacaccgatg ctgaccgttc
tcctattttt ctccagttta ttgattgtgt gtggcaaatg tcaaaacagt tccctacagc
ttttgaattc aatgaacaat ttttgattat aattttggat catctgtata gttgccgatt
tggtactttc ttattcaact gtgaatctgc tcgagaaaga cagaaggtta cagaaaggac
tgtttcttta tggtcactga taaacagtaa taaagaaaaa ttcaaaaacc ccttctatac
taaagaaatc aatcgagttt tatatccagt tgccagtatg cgtcacttgg aactctgggt
gaattactac attagatgga accccaggat caagcaacaa cagccgaatc cagtggagca
gcgttacatg gagctcttag ccttacgcga cgaatacata aagcggcttg aggaactgca
gctcgccaac tctgccaagc tttctgatcc cccaacttca ccttccagtc cttcgcaaat
gatgccccat gtgcaaactc acttctgagg ggggaccctg gcaccgcatt agagctcgaa
ataaaggcga tagctgactt tcatttgggg catttgtaaa aagtagatta aaatatttgc
ctccatgtag aacttgaact aacataatct taaactcttg aatatgtgcc ttctagaata
catattacaa gaaaactaca gggtccacac ggcaatcaga agaaaggagc tgagatgagg
ttttggaaaa ccctgacacc tttaaaaagc agtttttgaa agacaaaatt tagatttaat
ttacgtcttg agaaatacta tatatacaat atatattttg tgggcttaat tgaaacaaca
ttattttaaa atcaaagggg atatatgttt gtggaatgga ttttcctgaa gctgcttaac
agttgctttg gattctctaa gatgaatcca aatgtgaaag atgcatgtta ctgccaaaac
caaattgagc tcagcttcct aggcattacc caaaagcaag gtgtttaagt aattgccagc
ttttatacca tcatgagtgg tgacttaagg agaaatagct gtatagatga gtttttcatt
atttggaaat ttaggggtag aaaatgtttt cccctaattt tccagagaag cctattttta
tatttttaaa aaactgacag ggcccagtta aatatgattt gcatttttta aatttgccag
ttttattttc taaattcttt catgagcttg cctaaaattc ggaatggttt tcgggttgtg
gcaaacccca aagagagcac tgtccaagga tgtcgggagc atcctgctgc ttaggggaat
gttttcgcaa atgttgctct agtcagtcca gctcatctgc caaaatgtag ggctaccgtc
ttggatgcat gagctattgc tagagcatca tccttagaaa tcagtgcccc agatgtacat
gtgttgagcg tattcttgaa agtattgtgt ttatgcattt caatttcaat ggtgttggct
tcccctcccc accccacgcg tgcataaaaa ctggttctac aaatttttac ttgaagtacc
aggccgtttg ctttttcagg ttgttttgtt ttatagtatt aagtgaaatt ttaaatgcac
agttctattt gctatctgaa ctaattcatt tattaagtat atttgtaaaa gctaaggctc
gagttaaaac aatgaagtgt tttacaatga tttgtaaagg actatttata actaatatgg
ttttgttttc aatgaattaa gaaagattaa atatatcttt gtaaattatt ttatgtcata
gtttaattgg tctaccaagt aagacatctc aaatacagta gtataatgta tgaattttgt
aagtataaga aattttatta gacattctct tactttttgt aaatgctgta aatatttcat
aaattaacaa agtgtcactc cataaaaaga aagctaatac taatagccta aaagattttg
tgaaatttca tgaaaacttt ttaatggcaa taatgactaa agacctgctg taataaatgt
attaactgaa acctaaaaaa aaaaaaaaaa aa
mouse MTM1 protein sequence (NP_064310.1)
SEQ ID NO: 6
MASASASKYNSHSLENESIKKVSQDGVSQDVSETVPRLPGELLITEKEVIYICPFNGPIKGRVYI
TNYRLYLRSLETDSALILDVPLGVISRIEYMGGATSRGENSYGLDITCKDLRNLRFALKQEGHSR
RDMFEILVKHAFPLAHNLPLFAFVNEEKFNVDGWTVYNPVEEYRRQGLPNHHWRISFINKCYELC
ETYPALLVVPYRTSDDDLRRIATFRSRNRLPVLSWIHPENKMVIMRCSQPLVGMSGKRNKDDEKY
LDVIRETNKQTSKLMIYDARPSVNAVANKATGGGYESDDAYQNSELSFLDIHNIHVMRESLKKVK
DIVYPNIEESHWLSSLESTHWLEHIKLVLTGAIQVADQVSSGKSSVLVHCSDGWDRTAQLTSLAM
LMLDSFYRTIEGFEILVQKEWISFGHKFASRIGHGDKNHADADRSPIFLQFIDCVWQMSKQFPTA
FEFNEGFLITVLDHLYSCRFGTFLFNCDSARERQKLTERTVSLWSLINSNKDKFKNPFYTKEINR
VLYPVASMRHLELWVNYYIRWNPRVKQQQPNPVEQRYMELLALRDDYIKRLEELQLANSAKLADA
PASTSSSSQMVPHVQTHF
mouse MTM1 nucleic acid sequence (NM_019926.2)
SEQ ID NO: 7
ggtgagttcg ctttcttggc tgacctggct cggagccggg cattgcgggg atccaggatt
ggaaaggttc caggatggct tctgcatcag catctaagta taattcacac tccttggaga
atgaatccat taagaaagtg tctcaagatg gagtcagtca ggatgtgagt gagactgtcc
ctcggctccc aggggagtta ctaattactg aaaaagaagt tatttacata tgtcctttca
atggccccat taagggaaga gtttacatca caaattatcg tctttattta agaagtttgg
aaacggattc tgctctaata cttgatgttc ctctgggtgt gatatcaaga attgaatata
tgggaggcgc gactagtaga ggagaaaatt cctatggtct agatattact tgtaaagatt
tgagaaacct gaggtttgca ttgaagcaag aaggccacag cagaagagat atgtttgaga
tccttgtaaa acatgccttt cctctggcac acaatctgcc attatttgca tttgtaaatg
aagagaagtt taacgtggat gggtggactg tttataatcc agttgaagaa tatagaaggc
agggcctgcc caatcaccat tggaggataa gttttattaa caagtgctat gagctctgtg
agacataccc tgctcttttg gtggttccct atcggacctc agatgatgat cttaggagga
tcgcaacgtt tagatcccga aatcggcttc ctgtactgtc gtggattcac ccagaaaaca
aaatggtcat tatgcgctgc agtcagcctc ttgtcggtat gagtggtaaa agaaataaag
atgacgagaa atacctggat gtgatcaggg aaactaacaa acaaacttct aagctcatga
tttatgatgc acgacccagt gtaaatgcag tcgccaacaa ggcaacagga ggaggatatg
aaagtgatga cgcatatcaa aactcagaac tttccttctt agacattcat aatattcatg
ttatgcgaga atctttaaaa aaagtgaaag atattgttta tcccaacata gaagaatctc
attggttgtc cagtttggag tctactcatt ggttagaaca tatcaagctt gttctgaccg
gtgccattca agtggcagac caagtgtctt caggaaagag ctcggtactt gtgcactgca
gtgacggatg ggacaggacc gctcagctga catccttggc catgctgatg ttggacagct
tctacagaac tattgaaggc tttgagatat tggtacagaa agagtggata agttttggcc
ataaatttgc atctagaata ggtcatggtg ataaaaacca tgctgatgct gatcgatctc
ctatttttct tcagtttatt gactgtgtgt ggcagatgtc gaaacagttc cccacagctt
ttgagttcaa tgaaggcttt ttgattaccg ttttggatca tctgtatagc tgtcgatttg
gtactttctt attcaactgt gactcggctc gagaaagaca gaaacttaca gaaagaacag
tttctctatg gtcgctaatt aacagcaata aagacaaatt caaaaacccc ttctatacaa
aagaaatcaa tcgggttttg tatccagttg ccagcatgcg tcacttggaa ctgtgggtga
attattacat ccgatggaat cccagggtca agcagcaaca gcccaaccca gtggagcagc
gttacatgga gcttttggcc ttgcgtgacg attatataaa gaggctcgag gaattgcagc
tggccaactc cgccaagctt gctgatgccc ccgcttcgac ttccagttcg tcacagatgg
tgccccatgt gcagacgcac ttctgagggg actcacttct ggcactgcac ttgaactcta
gataagtgaa atagctgact ctcattctgg gcatgtggac aaagtagatt taaagtgtct
gcctccattt agaagttcaa ctaacatctt agacttttga gtatgtgcct tctgtaatac
atatcacaag aaatcgatgg tgtccgtgtg gcaatcataa ggaaggagtc aagagggggt
tctggaaaat cctcatactt ttttttacaa agcacttttg caaagataaa acttaaattt
aatttacctc tatataaatt ctacatatac agtatgtatt ttgtgggctt aattgaaata
ttattttaaa tccagggggg agatttgttt gcaaaatgta ttttcctcca gctgcttata
acagttgctt tggattatct aaaattaatc caaatgtgaa agatgggtat tactgccaaa
gccaaattgc actctgcttc ttcagcaaat tccaagagca aggcgtttaa ataattgcca
atttttattt taccataagt ggtaaggtaa aaagaaagat gaacatttca tcattttgaa
tttttgaaaa taaaaggttc tcccatcatt tttcaagaga agcacatttt tatattaaga
aaaagtgata aggtttgatt tttttttccc tcaacattct cagctttgct ttctaaatta
tcccatgatt tttgtctaac actgagtcat actcaggttg aaggaaaccc ataaatagca
ctgtgcgagg agctggctgg cttctgctgc ttagaggaat atgttcgcaa acatgcctct
agtcaattcg ccttatctgc tgaagtgtag gggcaccgcc ttgaatggat gagctatggc
tagagcatct ttctttacag taatgcccca ggtgtattct gtttatgtct ctctgtttaa
atggtgtgcg tgcataaaaa cttgctctgc acattattac ttgaagtact gggcaatttg
ctttttcagg ttttttttca ttttgttttg tagtatgaaa tggaatttta aatgcacagt
tctatttgat atccgaacta attcatttag taaatatatt tgtaaaagct aaagttaaat
caattaatgt tttacagtga tttgtaaagg attatttata gctaatatgg ttttgttttc
agtgaattaa gagagattac atttatcttt gtaaattatt ttatgtcata gcttaatggc
ctaccaaatg agacatctca aatataatag tataatgtat ggattttgta agtataaaaa
ttattagata ttcgtttgct ttttgtaaac actgtaaata tttcataaat taaaatgtgt
cactccataa gaagaaaaaa ctaatactaa tagttgacag gaattggtga aatttcatga
aaatattttc attgcaataa atattaaaag acctgctg
rat MTM1 protein sequence (NP_001013065.1)
SEQ ID NO: 8
MASSSASDCDAHPVERESMRKVSQDGVRQDMSKSGPRLPGESAITDKEVIYICPFSGPVKGRLYI
TNYRLYLRSLETDLAPILDVPLGVISRIEKMGGVTSRGENSYGLDITCKDLRNLRFALKQEGHSR
RDIFDVLTRHAFPLAYNLPLFAFVNEEKFKVDGWAIYNPVEEYRRQGLPDRHWRISFVNQRYELC
DTYPALLVVPYRASDDDLRRVATFRSRNRIPVLSWIHPENRAAIMRCSQPLVGVGGKRSRDDERY
LDIIRETNKQTSKLTIYDARPGVNAVANKATGGGYEGEDAYPHAELSFLDIHNIHVMRESLRRVR
DIVYPHVEEAHWLSSLESTHWLEHIKLLLTGAIRVADKVASGLSSVLVHCSDGWDRTAQLTTLAM
LMLDGFYRSIEGFEILVQKEWISFGHKFSSRIGHGDKNHADADRSPIFLQFIDCVWQMTKQFPTA
FEFNECFLVAILDHLYSCRFGTFLLNCEAARERQRLAERTVSVWSLINSNKDEFTNPFYARESNR
VIYPVTSVRHLELWVNYYIRWNPRIRQQQPHPM
rat MTM1 nucleic acid sequence (NM_001013047.1)
SEQ ID NO: 9
gcgagcgcgt tggcaccagc ggcccccgga gtctcaggtt ccaggatggc gtcctcgtca
gcctctgact gtgatgcaca ccccgtggag cgtgagtcca tgaggaaggt gtctcaagat
ggagtccgtc aggatatgag caagagtggg cctcgcctcc caggggaatc agccatcact
gacaaggaag tcatctacat ttgtcccttc agcggccccg taaagggacg actttacatc
accaattacc gtctctacct gagaagtctg gagacggact tggctccgat tcttgacgtc
cccctaggcg tgatatcgag aatagagaaa atgggaggcg tgacgagtcg aggagagaat
tcctacggcc ttgatatcac ctgcaaagac ctgaggaacc tgaggttcgc tctgaagcag
gaaggacaca gcaggaggga catctttgac gtcctcacca gacacgcctt ccccctggct
tacaacctgc cgttgtttgc attcgtgaac gaggagaagt ttaaagtgga tggatgggcg
atttacaacc cggttgaaga gtacagaagg cagggcctcc ccgatcgcca ttggcggata
agtttcgtca atcagcgcta cgagctctgt gacacctacc ctgccctcct ggtcgtcccc
taccgtgcgt ccgatgatga cctcagaaga gttgcaacct ttaggtccag aaaccggatt
cccgtgctgt cgtggatcca cccagagaac agggcggcga tcatgcggtg cagtcagcct
ctggttggtg tgggcgggaa gagaagcaga gatgatgaga gatacctgga catcatccgg
gaaaccaata agcagacctc gaagctcaca atttacgatg cgcggcccgg cgtcaatgcg
gtggccaaca aggcaacggg aggcggctat gagggcgagg acgcgtaccc tcacgcggag
ctctccttcc tggacatcca caacatccac gtgatgcggg aatccttacg gagggtgagg
gacatcgtgt acccccacgt ggaggaagct cattggctgt ccagcttgga gtccacccat
tggttagagc acatcaagct tctcctcact ggtgccatcc gggtcgcaga caaggtggca
tcggggctga gttcagtcct cgtgcactgc agtgacggct gggaccggac ggctcaactg
accacgctgg ccatgctgat gctcgatggc ttctaccgca gcatcgaggg ctttgagatc
ctggtgcaga aggagtggat cagcttcgga cacaagtttt catctagaat tggccacggt
gacaagaacc acgcggatgc cgaccgctcc ccgattttcc tgcagttcat cgactgcgtg
tggcagatga cgaagcagtt ccccacagct ttcgagttca acgagtgctt cctggttgcc
atcttggatc acctgtacag ctgccggttc gggactttct tactaaactg tgaggcggca
cgggagagac agagactcgc agaaaggacg gtgtctgtgt ggtccctgat caacagcaac
aaagacgaat tcacaaaccc gttctacgca agggagagca accgcgtgat ctacccggtc
accagcgtgc gccacctgga actgtgggtg aattactaca tccggtggaa ccccaggatc
cggcagcagc agccccaccc catgtagcag cgatataatg agctcctggc cctgcgtgac
gattacatca agaagctgga ggagctgcag ctggccacgc ccaccaagct cactgactcc
tccaccccgc cttccggttc cgcacagata gctccccgca tgcaaactca cttctgaggg
ggttccgggc cccaaaccct gaataagtga cgtcaccaac ttccgttctg tgcgcttgtg
caaaggggat ataaagtctc cgcctctgtg tagaagtcga actaacaccc tagaaccttg
tgtgacacgt gtgagtgtgc gccttttgtg acgtgtgagt gtgcgatttg tgtgacatgt
gtgaatgtgt accctgtgtg atacgtgcaa gtgtgcgcct tgtgtaaagt tcgtgagtgt
gcacctcctg taacatgttt tgcaaggaat ctactgcgct tgtgtgccag tcgtgagtac
agagtagggg gggtcccgga aaaatcctca cactttttta caaagcgctt gtgcaaagat
taaaattaaa ttatatcaat aattatataa attattataa ttatattgca aagattaaaa
agttaaattt agtttacctc tatataaatc cagacataca taatatgtac tctgtgcgct
taattgaaac gttattttaa atccagaggg gagatttttt ttgtaaaatg gatttttcct
ccagccactt attttgcaaa gataaaaaag ttaaaataaa agttaaattt aattataaaa
aaaaaaaaaa aaaaaaaaaa aaaaaaaaaa aaaaaaaaaa aaaaaaaaaa aaaaaaaaaa
aaaaaaaaaa aaaaaa
linker sequence “GSTS”
SEQ ID NO: 10
GSTSGSGKSSEGKG
Fv3E10-GSTS-hMTM1
SEQ ID NO: 11
DIVLTQSPASLAVSLGQRATISCRASKSVSTSSYSYMHWYQQKPGQPPKLLIKYASYLESGVPAR
FSGSGSGTDFHLNIHPVEEEDAATYYCQHSREFPWTFGGGTKLELKGGGGSGGGGSGGGSEVQLV
ESGGGLVKPGGSRKLSCAASGFTFSNYGMHWVRQAPEKGLEWVAYISSGSSTIYYADTVKGRFTI
SRDNAKNTLFLQMTSLRSEDTAMYYCARRGLLLDYWGQGTTLTVSSEQKLSEEDLGSTSGSGKSS
EGKGMASASTSKYNSHSLENESIKRTSRDGVNRDLTEAVPRLPGETLITDKEVIYICPFNGPIKG
RVYITNYRLYLRSLETDSSLILDVPLGVISRIEKMGGATSRGENSYGLDITCKDMRNLRFALKQE
GHSRRDMFEILTRYAFPLAHSLPLFAFLNEEKFNVDGWTVYNPVEEYRRQGLPNHHWRITFINKC
YELCDTYPALLVVPYRASDDDLRRVATFRSRNRIPVLSWIHPENKTVIVRCSQPLVGMSGKRNKD
DEKYLDVIRETNKQISKLTIYDARPSVNAVANKATGGGYESDDAYHNAELFFLDIHNIHVMRESL
KKVKDIVYPNVEESHWLSSLESTHWLEHIKLVLTGAIQVADKVSSGKSSVLVHCSDGWDRTAQLT
SLAMLMLDSFYRSIEGFEILVQKEWISFGHKFASRIGHGDKNHTDADRSPIFLQFIDCVWQMSKQ
FPTAFEFNEQFLIIILDHLYSCRFGTFLFNCESARERQKVTERTVSLWSLINSNKEKFKNPFYTK
EINRVLYPVASMRHLELWVNYYIRWNPRIKQQQPNPVEQRYMELLALRDEYIKRLEELQLANSAK
LSDPPTSPSSPSQMMPHVQTHFHHHHHH
Note-in SEQ ID NO: 11-linker sequences are underlined and epitope
tags are double underlined
variable heavy chain CDR1 of exemplary 3E10 molecule
SEQ ID NO: 12
NYGMH
variable heavy chain CDR2 of exemplary 3E10 molecule
SEQ ID NO: 13
YISSGSSTIYYADTVKG
variable heavy chain CDR3 of exemplary 3E10 molecule
SEQ ID NO: 14
RGLLLDY
variable light chain CDR1 of exemplary 3E10 molecule
SEQ ID NO: 15
RASKSVSTSSYSYMH
variable light chain CDR2 of exemplary 3E10 molecule
SEQ ID NO: 16
YASYLES
variable light chain CDR3 of exemplary 3E10 molecule
SEQ ID NO: 17
QHSREFPWT
Fv3E10-GSTS-hMTM1
SEQ ID NO: 18
SAGIHDIVLTQSPASLAVSLGQRATISCRASKSVSTSSYSYMHWYQQKPGQPPKLLIKYASYLES
GVPARFSGSGSGTDFHLNIHPVEEEDAATYYCQHSREFPWTFGGGTKLELKGGGGSGGGGSGGGS
EVQLVESGGGLVKPGGSRKLSCAASGFTFSNYGMHWVRQAPEKGLEWVAYISSGSSTIYYADTVK
GRFTISRDNAKNTLFLQMTSLRSEDTAMYYCARRGLLLDYWGQGTTLTVSSEQKLSEEDLGSTSG
SGKSSEGKGMASASTSKYNSHSLENESIKRTSRDGVNRDLTEAVPRLPGETLITDKEVIYICPFN
GPIKGRVYITNYRLYLRSLETDSSLILDVPLGVISRIEKMGGATSRGENSYGLDITCKDMRNLRF
ALKQEGHSRRDMFEILTRYAFPLAHSLPLFAFLNEEKFNVDGWTVYNPVEEYRRQGLPNHHWRIT
FINKCYELCDTYPALLVVPYRASDDDLRRVATFRSRNRIPVLSWIHPENKTVIVRCSQPLVGMSG
KRNKDDEKYLDVIRETNKQISKLTIYDARPSVNAVANKATGGGYESDDAYHNAELFFLDIHNIHV
MRESLKKVKDIVYPNVEESHWLSSLESTHWLEHIKLVLTGAIQVADKVSSGKSSVLVHCSDGWDR
TAQLTSLAMLMLDSFYRSIEGFEILVQKEWISFGHKFASRIGHGDKNHTDADRSPIFLQFIDCVW
QMSKQFPTAFEFNEQFLIIILDHLYSCRFGTFLFNCESARERQKVTERTVSLWSLINSNKEKFKN
PFYTKEINRVLYPVASMRHLELWVNYYIRWNPRIKQQQPNPVEQRYMELLALRDEYIKRLEELQL
ANSAKLSDPPTSPSSPSQMMPHVQTHFHHHHHH
Note-in SEQ ID NO: 18-linker sequences are underlined and epitope
tags are double underlined
SEQ ID NO: 19
AGIH
SEQ ID NO: 20
SAGIH

### INCORPORATION BY REFERENCE

All publications and patents mentioned herein are hereby incorporated by reference in their entirety as if each individual publication or patent was specifically and individually indicated to be incorporated by reference.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method of increasing muscle contractility in a subject having myotubular myopathy, comprising:
systemically administering to the subject an amount of a chimeric polypeptide according to a dosing regimen, wherein the chimeric polypeptide comprises:
(i) a myotubularin polypeptide and
(ii) an antibody or antibody fragment comprising
a heavy chain CDR1 having the amino acid sequence of SEQ ID NO: 12,
a heavy chain CDR2 having the amino acid sequence of SEQ ID NO: 13,
a heavy chain CDR3 having the amino acid sequence of SEQ ID NO: 14,
a light chain CDR1 having the amino acid sequence of SEQ ID NO: 15,
a light chain CDR2 having the amino acid sequence of SEQ ID NO: 16, and
a light chain CDR3 having the amino acid sequence of SEQ ID NO: 17,
wherein the administering of less than 20 doses of said chimeric polypeptide is effective to achieve an initial response, wherein the initial response comprises increasing muscle contractility in at least a subset of muscle in said subject by at least 50% relative to that observed prior to initiation of treatment with the chimeric polypeptide.

2. The method of claim 1, wherein the initial response comprises increasing muscle contractility in at least a subset of muscle in said subject by at least 100%.

3. The method of claim 2, wherein the initial response comprises increasing muscle contractility in at least a subset of muscle in said subject by at least 200%.

4. The method of claim 3, wherein the initial response comprises increasing muscle contractility in at least a subset of muscle in said subject by at least 300%.

5. The method of claim 4, wherein the initial response comprises increasing muscle contractility in at least a subset of muscle in said subject by at least 350%.

6. A method of increasing muscle contractility in a subject having myotubular myopathy, comprising:
systemically administering to a subject an effective amount of a chimeric polypeptide comprising:
(i) a myotubularin polypeptide and
(ii) an antibody or antibody fragment comprising
a heavy chain CDR1 having the amino acid sequence of SEQ ID NO: 12,
a heavy chain CDR2 having the amino acid sequence of SEQ ID NO: 13,
a heavy chain CDR3 having the amino acid sequence of SEQ ID NO: 14,
a light chain CDR1 having the amino acid sequence of SEQ ID NO: 15,
a light chain CDR2 having the amino acid sequence of SEQ ID NO: 16, and
a light chain CDR3 having the amino acid sequence of SEQ ID NO: 17,
wherein the subject receives a first dose of said chimeric polypeptide after the subject is 5 years of age.

7. The method of claim 6, wherein the subject receives a first dose of said chimeric polypeptide after the subject is 12 years of age.

8. The method of claim 7, wherein the subject receives a first dose of said chimeric polypeptide after the subject is 15 years of age.

9. The method of claim 8, wherein the subject receives a first dose of said chimeric polypeptide after the subject is 18 years of age.

10. A method of increasing muscle contractility in a subject having myotubular myopathy, comprising:
systemically administering to a subject an effective amount of a chimeric polypeptide comprising:
(i) a myotubularin polypeptide and
(ii) an antibody or antibody fragment comprising
a heavy chain CDR1 having the amino acid sequence of SEQ ID NO: 12,
a heavy chain CDR2 having the amino acid sequence of SEQ ID NO: 13,
a heavy chain CDR3 having the amino acid sequence of SEQ ID NO: 14,
a light chain CDR1 having the amino acid sequence of SEQ ID NO: 15,
a light chain CDR2 having the amino acid sequence of SEQ ID NO: 16, and
a light chain CDR3 having the amino acid sequence of SEQ ID NO: 17,
wherein the subject receives a first dose of said chimeric polypeptide before the subject is 5 years of age.

11. The method of claim 10, wherein the subject receives a first dose of said chimeric polypeptide before the subject is 1 year of age.

12. The method of claim 11, wherein the subject receives a first dose of said chimeric polypeptide before the subject is 9 months of age.

13. The method of claim 12, wherein the subject receives a first dose of said chimeric polypeptide before the subject is 6 months of age.

14. The method of claim 13, wherein the subject receives a first dose of said chimeric polypeptide before the subject is 3 months of age.

15. The method of any of claims 6-14, wherein the administering of less than 20 doses of said chimeric polypeptide is effective to achieve an initial response, wherein the initial response comprises increasing muscle contractility in at least a subset of muscle in said subject by at least 50% relative to that observed prior to initiation of treatment with the chimeric polypeptide.

16. The method of claim 15, wherein the initial response comprises increasing muscle contractility in at least a subset of muscle in said subject by at least 100%.

17. The method of claim 15, wherein the initial response comprises increasing muscle contractility in at least a subset of muscle in said subject by at least 200%.

18. The method of claim 15, wherein the initial response comprises increasing muscle contractility in at least a subset of muscle in said subject by at least 300%.

19. The method of any of claims 1-15, wherein the administering of less than 10 doses of said chimeric polypeptide is effective to achieve an initial response, wherein the initial response comprises increasing muscle contractility in at least a subset of muscle in said subject by at least 50% relative to that observed prior to initiation of treatment with the chimeric polypeptide

20. The method of any of claims 1-19, wherein increasing muscle contractility in at least a subset of muscle in said subject is effective to improve respiratory function in said subject.

21. The method of any of claims 1-19, wherein increasing muscle contractility in at least a subset of muscle in said subject is effective to increase mobility in said subject.

22. A method of increasing muscle contractility in a subject having myotubular myopathy, comprising:
systemically administering to the subject 4-20 doses of a chimeric polypeptide comprising:
(i) a myotubularin polypeptide and
(ii) an antibody or antibody fragment comprising
a heavy chain CDR1 having the amino acid sequence of SEQ ID NO: 12,
a heavy chain CDR2 having the amino acid sequence of SEQ ID NO: 13,
a heavy chain CDR3 having the amino acid sequence of SEQ ID NO: 14,
a light chain CDR1 having the amino acid sequence of SEQ ID NO: 15,
a light chain CDR2 having the amino acid sequence of SEQ ID NO: 16, and
a light chain CDR3 having the amino acid sequence of SEQ ID NO: 17,
wherein prior to said administration of said chimeric polypeptide, said subject has muscle contractility that is less than 5% of muscle contractility in a healthy control subject; and;
wherein, following the administration of said 4-20 doses of said chimeric polypeptide, said muscle contractility in said subject is at least 10% of the muscle contractility in the healthy control subject.

23. The method of claim 22, wherein, following the administration of said 4-20 doses of said chimeric polypeptide, said muscle contractility in said subject is at least 15% of the muscle contractility in the healthy control subject.

24. The method of claim 23, wherein, following the administration of said 4-20 doses of said chimeric polypeptide, said muscle contractility in said subject is at least 18% of the muscle contractility in the healthy control subject.

25. The method of any of claims 1-24, wherein the method increases skeletal muscle contractility.

26. The method of claim 25, wherein said skeletal muscle comprises Type I muscle fibers.

27. The method of claim 25, wherein said skeletal muscle comprises Type II muscle fibers.

28. The method of claim 27, wherein said Type II muscle fibers are Type IIa muscle fibers.

29. The method of claim 27, wherein said Type II muscle fibers are Type IIb muscle fibers.

30. The method of claim 27, wherein the Type II muscle fibers are Type IIx muscle fibers.

31. The method of any of claims 1-30, wherein the method decreases the subject's reliance on a respirator.

32. The method of any of claims 1-31, wherein the method increases diaphragm muscle contractility.

33. The method of any of claims 1-32, wherein the method increases facial muscle contractility.

34. The method of claim 33, wherein the method increases one or more of eyelid, jaw, tongue lips, mouth or throat muscle contractility.

35. The method of any of claims 1-34, wherein the method increases paraspinal muscle contractility.

36. The method of any of claims 1-35, wherein the method increases erector spinae muscle contractility.

37. The method of any of claims 1-36, wherein the method increases lower limb muscle contractility.

38. The method of any of claims 1-37, wherein the method increases upper limb muscle contractility.

39. The method of any one of claims 1-38, wherein the chimeric polypeptide is formulated with a pharmaceutically acceptable carrier.

40. The method of any one of claims 1-39, wherein the chimeric polypeptide is administered parenterally.

41. The method of any one of claims 1-40, wherein the chimeric polypeptide is administered intravenously.

42. The method of any one of claims 1-40, wherein the chimeric polypeptide is administered subcutaneously.

43. The method of any of claims 1-40, wherein the chimeric polypeptide is administered intramuscularly.

44. The method of any one of claims 1-43, wherein the method comprises administering at least 6 doses of chimeric polypeptide to said subject.

45. The method of claim 44, wherein the method comprises administering at least 10 doses of chimeric polypeptide to said subject.

46. The method of claim 45, wherein the method comprises administering at least 20 doses of chimeric polypeptide to said subject.

47. The method of any of claim 1-5 or 15-46, wherein the method further comprises administering one or more additional doses of chimeric polypeptide after achieving said initial response.

48. The method of claim 47, wherein administration of said one or more additional doses substantially maintains the initial response.

49. The method of claim 47, wherein administration of said one or more additional doses provides further improvement relative to the initial response.

50. The method of any one of claims 1-49, wherein the method comprises administering chimeric polypeptide to said subject throughout the lifetime of said subject.

51. The method of any one of claims 1-50, wherein the method comprises administering chimeric polypeptide to said subject until said subject is asymptomatic for myotubular myopathy.

52. The method of any of claims 1-51, wherein the method comprises administering the chimeric polypeptide to said subject at least once over a two week period.

53. The method of claim 52, wherein the method comprises administering the chimeric polypeptide to said subject at least once over a one week period.

54. The method of claim 53, wherein the method comprises administering the chimeric polypeptide to said subject at least twice over a one week period.

55. The method of claim 54, wherein the method comprises administering the chimeric polypeptide to said subject at least once a day.

56. The method of any one of claims 1-55, wherein said antibody or antibody fragment is chimeric or humanized.

57. The method of any of claims 1-56, wherein the chimeric polypeptide comprises an scFv.

58. The method of any of claims 1-57, wherein the chimeric polypeptide is a fusion protein.

59. The method of any of claims 1-58, wherein the chimeric polypeptide comprises the myotubularin polypeptide comprising the amino acid sequence of SEQ ID NO: 1.

60. The method of any of claims 1-59, wherein the chimeric polypeptide comprises the antibody or antibody fragment comprising a heavy chain variable domain comprising the amino acid sequence of SEQ ID NO: 2, or a humanized antibody or antibody fragment thereof.

61. The method of any of claims 1-60, wherein the chimeric polypeptide comprises the antibody or antibody fragment comprising a light chain variable domain comprising the amino acid sequence of SEQ ID NO: 4, or a humanized antibody or antibody fragment thereof.

62. The method of any of claims 1-61, wherein the chimeric polypeptide comprises the amino acid sequence set forth in SEQ ID NO: 11, in the presence or absence of one or more epitope tags.

63. The method of claim 62, wherein the chimeric polypeptide comprises the amino acid sequence set forth in SEQ ID NO: 18, in the presence or absence of one or more epitope tags.
